(19) United States
(12) Patent Application Publication
(10) Pub. No.: US 2009/0171203 A1
(43) Pub. Date: Jul. 2, 2009

(54) Cryotherapy Insertion System and Method

(76) Inventors: Ofer Avital, Yokneam Ilit (IL); Eyal Kochavi, Haifa (IL); Amir Pansky, Atlit (IL); Yaron Hefetz, Herzlia (IL); Pazit Pianka, Kochav Yair (IL)

Correspondence Address: MARTIN D. MOYNIHAN d/b/a PRTSI, INC., P.O. BOX 16446, ARLINGTON, VA, 22215, US

(21) Appl. No.: 12/226,949

(22) PCT Filed: May 2, 2007
(86) PCT No.: PCT/IL2007/000539

§ 371 Date: Feb. 18, 2009

Related U.S. Application Data
(60) Provisional application No. 60/796,519, filed on May 2, 2006.

(51) Int. Cl. A61B 18/02; A61B 19/00; A61B 8/12

(52) U.S. Cl. 600/439; 606/21; 606/130

(57) ABSTRACT

Methods and systems for inserting therapeutic probes into a patient are present. Embodiments include an automated probe insertion system and a probe insertion guide comprising a moveable probe guide sleeve; a positioner for positioning said probe guide sleeve; and a controller operable to receive a definition of a locus within a body of a patient and to direct positioning and orienting of said probe guide sleeve so that a probe advanced through said sleeve will advance in direction of said locus. Cryosurgery applications are presented.

### RELATED APPLICATIONS

This Application claims the benefit under 119(e) of U.S. Provisional Patent Application No. 60/796,519 filed May 2, 2006, the contents of which are incorporated herein by reference.

This application is related to U.S. application Ser. No. 11/219,648, the disclosure of which is incorporated herein by reference.

This application is related to two other PCT applications being filed on even date with this application in the Israel Receiving Office having the titles PROBE INSERTION GUIDE WITH USER-DIRECTING FEATURES and CRYOTHERAPY PLANNING AND CONTROL SYSTEM, and Attorney docket Nos. 39261 and 33982, and sharing applicant Galil Medical Ltd. with this Application, the disclosures of which are incorporated herein by reference.

### FIELD AND BACKGROUND OF THE INVENTION

The present invention relates to systems and methods for inserting a plurality of cryoprobes into a body of a patient.

U.S. Pat. No. 6,142,991 to Schatzberger teaches an apparatus which comprises (a) a plurality of cryosurgical probes of small diameter, the probes serve for insertion into the patient's organ, the probes being for producing ice-balls for locally freezing selected portions of the organ; (b) a guiding element including a net of apertures for inserting the cryosurgical probes therethrough; and (c) an imaging device for providing a set of images, the images being for providing information on specific planes located at specific depths within the organ, each of said images including a net of marks being correlated to the net of apertures of the guiding element, wherein the marks represent the locations of ice-balls which may be formed by the cryosurgical probes when introduced through said apertures of the guiding element to said distinct depths within the organ.

U.S. Pat. No. 6,905,492 to Zvuloni et al., and pending U.S. patent application Ser. No. 11/219,648, also by Zvuloni et al., which are is incorporated herein by reference, teach a system and method for planning a cryoablation procedure by simulating such a procedure based on preparatory imaging of a target site in a patient, by simulating the procedure, by recommending procedural steps and by evaluating procedural steps specified by a user. Zvuloni teaches use of integrated images displaying, in a common virtual space, a three-dimensional model of a surgical intervention site based on digitized preparatory images of the site from first imaging modalities, simulation images of cryoprobes used according to an operator-planned cryoablation procedure at the site, and real-time images provided by second imaging modalities during cryoablation. Zvuloni further teaches system-supplied recommendations for and evaluations of the planned cryoablation procedure, and system-supplied feedback to an operator and system-supplied guidance and control signals for operating a cryosurgery tool during cryoablation.

Background material relevant to planning cryosurgery is to be found in “Computerized Planning for Multiprobe Cryosurgery using a Force-field analogy” by David C. Lung, Thomas Stahovich and Yoed Rabin, in Computer Methods in Biomechanics and Biomedical Engineering, Vol. 7 No. 2, April 2004, pp. 101-110.

Background material relevant to planning cryosurgery is also to be found in “An efficient numerical technique for bioheat simulations and its application to computerized cryosurgery planning”, by Michael Rossi, Daigo Tanaka, Kenji Shimada, and Yoed Rabin in Computer Methods and Programs in Biomedicine 85 (2007) pp. 41-50.

### SUMMARY OF THE INVENTION

The present invention relates to a system and method for inserting a plurality of therapeutic probes, such as cryoprobes, into a body of a patient. Some embodiments provide guidance and assistance to a user during manual insertion of probes. Some embodiments provide fully automated insertion and removal of a plurality of probes.

Embodiments of the invention successfully address disadvantages of presently known configurations by providing improved efficiency and improved accuracy in cryoprobe insertion and highly automated procedures for terminating a cryotherapy operation.

Embodiments of the invention further successfully address disadvantages of presently known configurations by providing means and method for accurate insertion of probes from a variety of angles, thereby providing a freedom of probe insertion direction not provided by prove insertion guides known to prior art.

Embodiments of the invention further successfully address disadvantages of presently known configurations by lowering the level of specific expertise required of practitioners of cryotherapy, by automatic execution of a portion of cryotherapy procedure.

Embodiments of the invention further successfully address disadvantages of presently known configurations by reducing probability of human error, by automatic execution of a portion of cryotherapy procedure.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

FIG. 1 is a simplified block diagram of a planning system for planning a cryoablation procedure, according to methods of prior art;

FIGS. 2a and 2b are a flow chart showing a method for automatically generating a recommendation relating to a cryoablation procedure, according to methods of prior art;

FIG. 3a is a simplified block diagram of a system for facilitating a cryosurgery ablation procedure, according to methods of prior art;

FIG. 3b is a schematic diagram of mechanisms for control of cryosurgical tools by a surgical facilitation system, according to methods of prior art;

FIG. 4 is a simplified schematic of a system for planning and performing cryoablation, according to an embodiment of the present invention;

FIG. 5 is a simplified flowchart of a method for planning and managing a surgical intervention, according to an embodiment of the present invention;

FIG. 6a is a raw ultrasound image of a prostate its vicinity; and

FIG. 6b is a sample user input screen including the image of FIG. 6a after annotation by a user, according to an embodiment of the present invention;

FIG. 6c is a sample user input screen of FIG. 6b, further showing predicted isotherms and recommended probe locations, according to an embodiment of the present invention;

FIGS. 7a, 7b, and 7c are simplified schematics comparing differences in ablation volume contours produced by synchronized cooling of probes, anti-synchronized cooling of probes, and cooling of a probe while heating a neighboring probe respectively, according to an embodiment of the present invention;

FIG. 8 is simplified schematic of a cryotherapy system for assisted and/or automated insertion of therapeutic probes, according to an embodiment of the present invention;

FIG. 9 is a simplified schematic showing details of a probe gripper, according to an embodiment of the present invention;

FIG. 10 is a simplified schematic of a probe inserter, according to an embodiment of the present invention;

FIG. 11 is a simplified schematic of a “dual angle” configuration of a positioner, according to an embodiment of the present invention;

FIG. 12 is a simplified schematic of a “polar angle” configuration of a positioner, according to an embodiment of the present invention;

FIG. 13 is a simplified schematic of a “Cartesian” configuration of a positioner, according to an embodiment of the present invention;

FIG. 14 is a simplified schematic of a sterilization cover for a probe, according to an embodiment of the present invention; and

FIG. 15 is a simplified flow chart of a method for assisted and automated insertion of therapeutic probes into a patient, according to an embodiment of the present invention.

### DESCRIPTION OF THE PREFERRED EMBODIMENTS

The present invention relates system and method for inserting a plurality of therapeutic probes, such as cryoprobes, into a body of a patient. Specifically, embodiments of the present invention can be used to facilitate and/or partially or wholly automate processes of probe insertion and removal, by providing automated sequential insertion of operating tips of a plurality of cryoprobes into a plurality of loci defined within a cryosurgical ablation target. Some embodiments of the invention also provide means for defining said loci. In some embodiments, sensing probes or probes designed to protect vital organs may also be automatically inserted.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is further to be understood that some aspects of the present invention are presented hereinbelow in the context of discussions of an exemplary utilization, yet it is to be understood that the context of the examples provided is exemplary only, and not to be regarded as limiting.

In discussion of the various figures described hereinbelow, like numbers refer to like parts. The drawings are generally not to scale

For clarity, non-essential elements are omitted from some of the drawings.

To enhance clarity of the following descriptions, the following terms and phrases will first be defined:

The phrases “heat exchanger” and “heat-exchanging configuration” are used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when expanded through a Joule-Thomson heat exchanger. As is well known in the art, when gasses such as argon, nitrogen, air, krypton, CO₂, CF₄, and xenon, and various other gasses, at room temperature or colder, pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which, when passed at room temperature or warmer through a Joule-Thomson heat exchanger, have the property of becoming hotter. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating. A Joule-Thomson heater/cooler is thus a “Joule-Thomson heat exchanger” as defined above.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

The term “registration” as applied to images, physical systems and three-dimensional models in virtual space refers to processes of ascertaining relationships between positions, orientations, and scale of said images, physical systems and three-dimensional models so as to enable to relate distances and dimensions in one of said elements to distances and dimensions in others of said elements

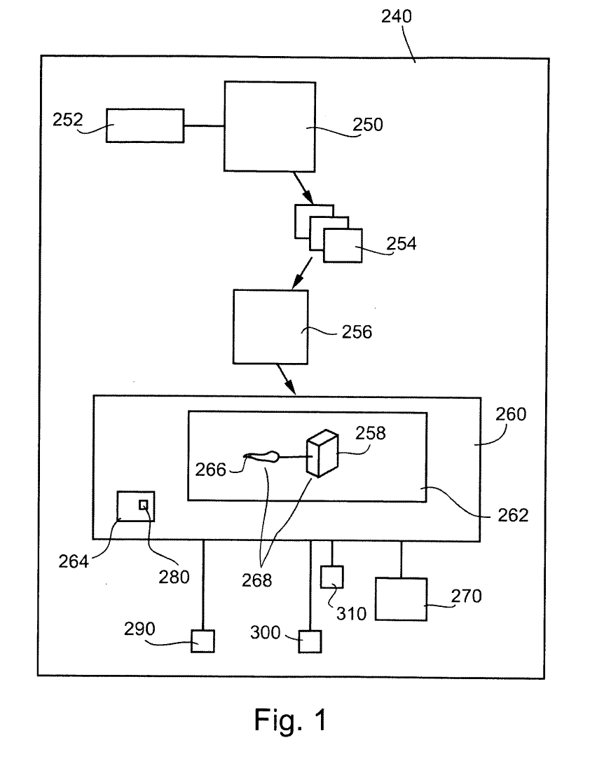

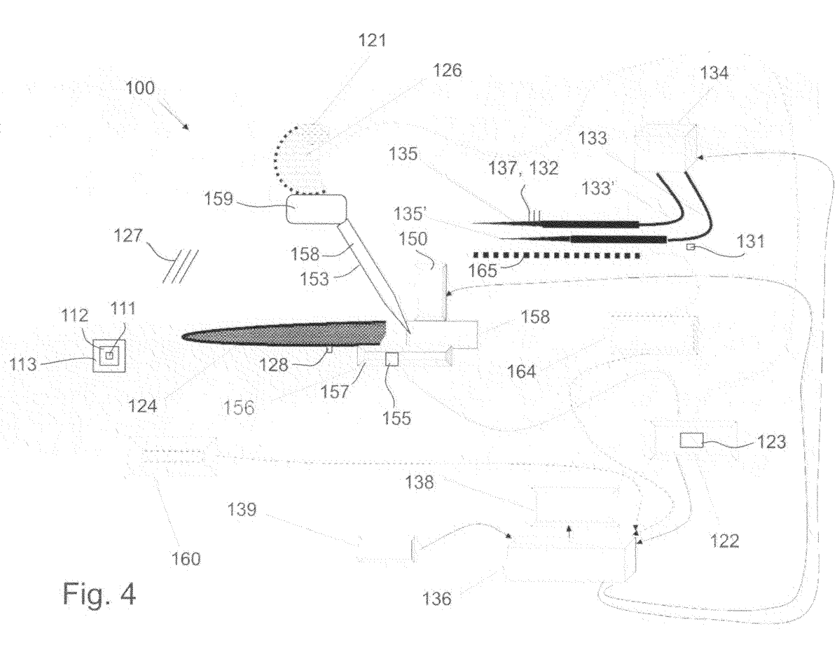

For purposes of better understanding the present invention, as illustrated in FIGS. 4-15 of the drawings, reference is first made to the construction and operation of a conventional (i.e., prior art) surgical planning system, as illustrated in FIGS. 1-3.

Reference is now made to FIG. 1, which is a simplified block diagram of a planning system for planning a cryoablation procedure, according to methods of prior art.

In FIG. 1, a planning system 240 for planning a cryoablation procedure comprises a first imaging modality 250 which serves for creating digitized preparatory images 254 of a cryoablation intervention site. First imaging modality 250 will typically be a magnetic resonance imaging system (MRI), an ultrasound imaging system, a computerized tomography imaging system (CT), a combination of these systems, or a similar system able to produce images of the internal tissues and structures of the body of a patient. First imaging modality 250 is for producing digitized images of a cryoablation intervention site, which site includes body tissues whose cryoablation is desired (referred to herein as “target” tissue), which may be a tumor or other structure, and body tissues and structures in the immediate neighborhood of the target tissues, which constitute the target tissue's physical environment.

Some types of equipment useable as first imaging modality 250, a CT system for example, typically produce a digitized image in a computer-readable format. If equipment used as first imaging modality 250 does not intrinsically produce digitized output, as might be the case for conventional x-ray imaging, then an optional digitizer 252 may be used to digitize non-digital images, to produce digitized preparatory images 254 of the site.

Digitized images 254 produced by first imaging modality 250 and optional digitizer 252 are passed to a three-dimensional modeler 256 for creating a three-dimensional model 258 of the intervention site. Techniques for creating a three dimensional model based on a set of two dimensional images are well known in the art. In the case of CT imaging, creation of a three dimensional model is typically an intrinsic part of the imaging process. PROVISION (http://www.algotec.com/web/products/provision.htm), from Algotec Inc., a division of Eastman Kodak Inc. based in Raanana, Israel, is an example of software designed to make a 2-D to 3-D conversion for images generated by CT scans. To accomplish the same purpose starting from ultrasound imaging, SONOReal™ software from BIOMEDICOM (http://www.biomedicom.com/) may be used.

Three dimensional model 258 is preferably expressible in a three dimensional Cartesian coordinate system.

Three dimensional model 258 is useable by a simulator 260 for simulating a cryosurgical intervention. Simulator 260 comprises a displayer 262 for displaying views of model 258, and an interface 264 useable by an operator for specifying loci for insertion of simulated cryoprobes 266 and operational parameters for operation of simulated cryoprobes 266 for cryoablating tissues. Thus, an operator (i.e., a user) can use simulator 260 to simulate a cryoablation intervention, by using interface 264 to command particular views of model 258, and by specifying both where to insert simulated cryoprobes 266 into an organ imaged by model 258, and how to operate cryoprobes 266. Typically, an operator may specify positions for a plurality of simulated cryoprobes 266, and further specify operating temperatures and durations of cooling for cryoprobes 266. Display 262 is then useable for displaying in a common virtual space an integrated image 268 comprising a display of three dimensional model 258 and a virtual display of simulated cryoprobes 266 inserted at said operator-specified loci.

Planning system 240 optionally comprises a memory 270, such as a computer disk, for storing operator-specified loci for insertion of cryoprobes and operator-specified parameters for operation simulated cryoprobes 266.

Interface 264 comprises a highlighter 280 for highlighting, under control of an operator, selected regions within three dimensional model 258. Operator-highlighted selected regions of model 258 are then optionally displayed as part of an integrated image 268.

In particular, highlighter 280 is useable by an operator for identifying tissues to be cryoablated. Preferably, interface 264 permits an operator to highlight selected regions of three dimensional model 258 so as to specify therein tissues to be cryoablated, or alternatively interface 264 permits an operator to highlight selected regions of digitized preparatory images 254, specifying therein tissues to be cryoablated. In the latter case, three-dimensional modeler 256 is then useable to translate regions highlighted on digitized preparatory images 254 into equivalent regions of three dimensional model 258. In both cases, tissues highlighted and selected to be cryoablated can be displayed by displayer 262 as part of integrated image 268, and can be recorded by memory 270 for future display or other uses.

Similarly, highlighter 280 is useable by an operator for identifying tissues to be protected from damage during cryoablation. Typically, important functional organs not themselves involved in pathology may be in close proximity to tumors or other structures whose destruction is desired. For example, in the case of cryoablation in a prostate, nerve bundles, the urethra, and the rectum may be in close proximity to tissues whose cryoablation is desired. Thus, highlighter 280 is useable by an operator to identify (i.e., to specify the location of) such tissues and to mark them as requiring protection from damage during cryoablation.

Preferably, interface 264 permits an operator to highlight selected regions of three dimensional model 258 so as to specify therein tissues to be protected from damage during cryoablation. Alternatively, interface 264 permits an operator to highlight selected regions of digitized preparatory images 254, specifying therein tissues to be protected during cryoablation. In the latter case, three-dimensional modeler 256 is then useable to translate regions highlighted on digitized preparatory images 254 into equivalent regions of three dimensional model 258. In both cases, tissues highlighted and selected to be protected from damage during cryoablation can be displayed by displayer 262 as part of integrated image 268, and can be recorded by memory 270 for future display or other uses.

Planning system 240 further optionally comprises a predictor 290, an evaluator 300, and a recommender 310.

Predictor 290 serves for predicting the effect on tissues of a patient, if a planned operation of cryoprobes 266 at the operator-specified loci is actually carried out according to the operator-specified operational parameters. Predictions generated by predictor 290 may optionally be displayed by displayer 262 as part of integrated image 268, in the common virtual space of image 268.

In a preferred embodiment, predictions of predictor 290 are based on several sources. The laws of physics, as pertaining to transfer of heat, provide one predictive source. Methods of calculation well known in the art may be used to calculate, with respect to any selected region within three dimensional model 258, a predicted temperature, given known locations of cryoprobes 266 which are sources of cooling in proximity to such a region, known temperatures and cooling capacities of cryoprobes 266, and a duration of time during which cryoprobes 266 are active in cooling. Thus, a mathematical model based on known physical laws allows to calculate a predicted temperature for any selected region within model 258 under operator-specified conditions.

Experimentation and empirical observation in some cases indicate a need for modifications of a simple mathematical model based on physical laws concerning the transfer of heat, as would be the case, for example, in a tissue wherein cooling processes were modified by a high rate of blood flow. However, methods for adapting such a model to such conditions are also well known in the art. Such methods take into account heat dissipation in flowing systems, affected by the flow.

An additional basis for predictions of predictor 290 is that of clinical observation over time. Table 1 provides an example of a predictive basis derived from clinical observation, relating to medium-term and long-term effects of cryoablation procedures in a prostate. The example provided in Table 1 relates to treatment of BPH by cryoablation under a standardized set of cryoprobe operating parameters.

TABLE 1
Predicted long-term effects of cryoablation

| Distance between | 3 week volume | 3 months volume |
|---|---|---|
| probes (mm) | consumption (%) | consumption (%) |
| 10 | 70 | 100 |
| 15 | 55 | 85 |
| 20 | 40 | 70 |
| 25 | 30 | 50 |

As may be seen from Table 1, clinical observation leads to the conclusion that reduction in the volume of a prostate following cryoablation is a gradual process which continues progressively for a number of weeks following a cryoablation procedure. The clinically derived information of Table 1, and similar clinically derived information, can also serve as a basis for predictions generated by predictor 290, and displayed by displayer 262 as part of integrated image 268 in the common virtual space of image 268.

Evaluator 300 is useable to compare results predicted by predictor 290 to goals of a surgical intervention as expressed by an operator. In particular, evaluator 300 can be used to compare intervention results predicted by predictor 290 under a given intervention plan specified by an operator, with that operator's specification of tissues to be cryoablated. Thus, an operator may use interface 264 to specify tissues to be cryoablated, plan an intervention by using interface 264 to specify loci for insertion of cryoprobes 266 and to specify a mode of operation of cryoprobes 266, and then utilize predictor 290 and evaluator 300 to predict whether, under his specified intervention plan, his/her goal will be realized and all tissues desired to be cryoablated will in fact be destroyed. Similarly, an operator may utilize predictor 290 and evaluator 300 to predict whether, under his/her specified intervention plan, tissues which he specified as requiring protection from damage during cryoablation will in fact be endangered by his planned intervention.

Recommender 310 may use predictive capabilities of predictor 290 and evaluator 300, or empirically based summaries of experimental and clinical data, or both, to produce recommendations for cryoablation treatment.

As discussed above, predictor 290 and evaluator 300 can be used to determine, for a given placement of a given number of cryoprobes and for a given set of operating parameters, whether a planned cryoablation procedure can be expected to be successful, success being defined as destruction of tissues specified as needing to be destroyed, with no damage or minimal damage to tissues specified as needing to be protected during cryoablation. Based on this capability, recommender 310 can utilize a variety of calculation techniques well known in the art to evaluate a plurality of competing cryoablation intervention strategies and to express a preference for that strategy which is most successful according to these criteria.

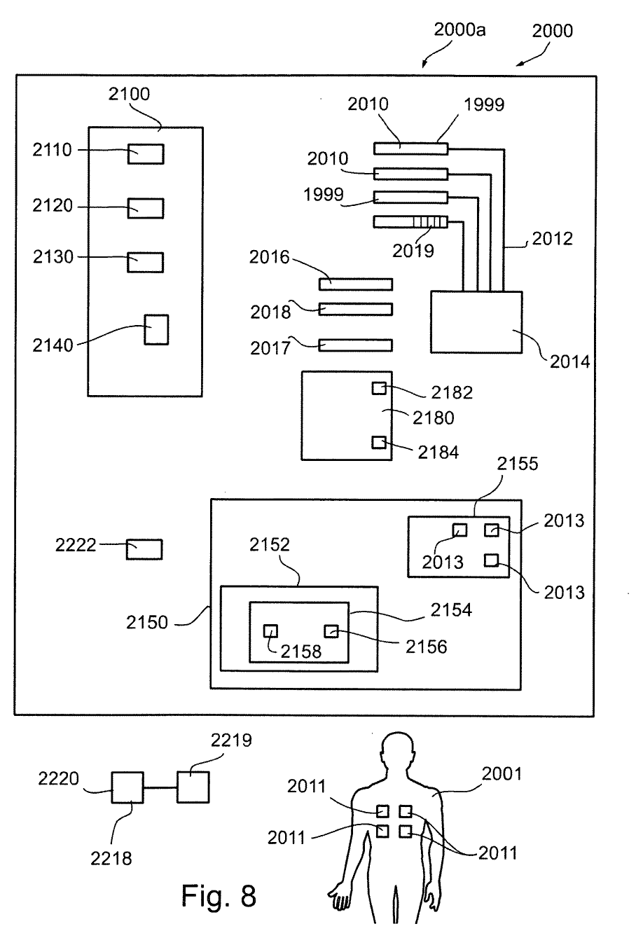

In particular, recommender 310 may consider several intervention strategies proposed by an operator, and recommend the most successful among them. Alternatively, an operator might specify a partial set of operating parameters, and recommender 310 might then vary (progressively or randomly) additional operating parameters to find a ‘best fit’ solution. For example, an operator might specify tissues to be destroyed, tissues to be protected, and a two-dimensional array of cryoprobes such as, for example, the two dimensional placement array of cryoprobes determined by the use of guiding element 115 having a net of apertures 120 shown in FIG. 8 hereinabove. Recommender 310 could then test a multitude of options for displacements of a set of cryoprobes in a third (depth) dimension to determine the shallowest and deepest penetration desirable for each cryoprobe. Recommender 310 could further be used to calculate a temperature and duration of freezing appropriate for each cryoprobe individually, or for all deployed cryoprobes controlled in unison, in a manner designed to destroy all tissues specified to be destroyed, while maximizing protection of tissues specified to be protected.

Recommendation activity of recommender 310 may also be based on empirical data such as experimental results or clinical results. Table 2 provides an example of a basis for making recommendations derived from clinical observation.

TABLE 2
Recommended number of cryoprobes to treat BPH

| American | | | |
|---|---|---|---|
| Urologists | Number of | | |
| Association | cross-sections | | |
| Questionnaire | with stricture of | Prostate | Number |
| Score | the Urethra | Volume | of probes |
| 0-7 | 1-3 | 25 | 2 |
| 0-7 | 1-3 | 40 | 2 |
| 0-7 | 2-5 | 40 | 2 |
| 0-7 | 1-3 | 50 | 2-3 |
| 0-7 | 2-5 | 50 | 2-3 |
| 0-7 | 1-3 | 60 | 2-3 |
| 0-7 | 2-5 | 60 | 3 |
| 0-7 | 2-5 | 100 | 4 |
| 8-19 | 1-3 | 40 | 2-3 |
| 8-19 | 2-5 | 40 | 2-3 |
| 8-19 | 1-3 | 50 | 2 |
| 8-19 | 2-5 | 50 | 2-3 |
| 8-19 | 1-3 | 60 | 3 |
| 8-19 | 2-5 | 60 | 3-4 |
| 8-19 | 2-5 | 100 | 4 |
| 20-35 | 1-3 | 40 | 3 |
| 20-35 | 2-5 | 40 | 3 |
| 20-35 | 1-3 | 50 | 4 |
| 20-35 | 2-5 | 50 | |
| 20-35 | 1-3 | 60 | 4 |
| 20-35 | 2-5 | 60 | 5 |
| 20-35 | 2-5 | 100 | 6 |

Table 2 relates to the treatment of BPH by cryoablation. Table 2 is essentially a table of expert opinion, wherein three criterions for describing the symptomatic state of a patient are related, by experts, to a recommendation for treatment. Table 2 was in fact compiled by a group of experts in the practice of cryoablation utilizing a particular tool, specifically a tool similar to that described in FIG. 8 hereinabove, yet a similar table may be constructed by other experts and for other tools. Moreover, feedback from the collective clinical experience of a population of users of a particular tool may be collected over time, for example by a company marketing such a tool or by an independent research establishment, and such collected information may be fed back into recommender 310 to build a progressively better informed and increasingly useful and reliable recommendation system.

The first column of Table 2, the AUA score, is the score of a questionnaire in use by the American Urological Association which may be found in Tanagho E. A., and McAninich J. W., Smith's General Urology, published by McGraw-Hill, Chapter 23. The AUA score is an estimate of severity of symptoms as subjectively reported by a patient, and relates to such urinary problems as incomplete emptying of the bladder, frequency of urination, intermittency, urgency, weak stream, straining, nocturia, and the patient's perceived quality of life as it relates to his urinary problems.

The second and third columns of Table 2 relate to diagnostic criteria discernable from three-dimensional model 258 or from digitized preparatory images 254 from which model 258 derives. The second column is a measure of the length of that portion of the urethra observed to be constricted by pressure from a patient's prostate. The third column is a measure of the volume of that patient's prostate. Table 2 constitutes a basis for recommending an aspect of a cryoablation treatment for BPH, specifically for recommending, in column four, an appropriate number of cryoprobes to be used in treating a specific patient, based on three quantitative evaluations of his condition constituted by the columns one, two and three of Table 2.

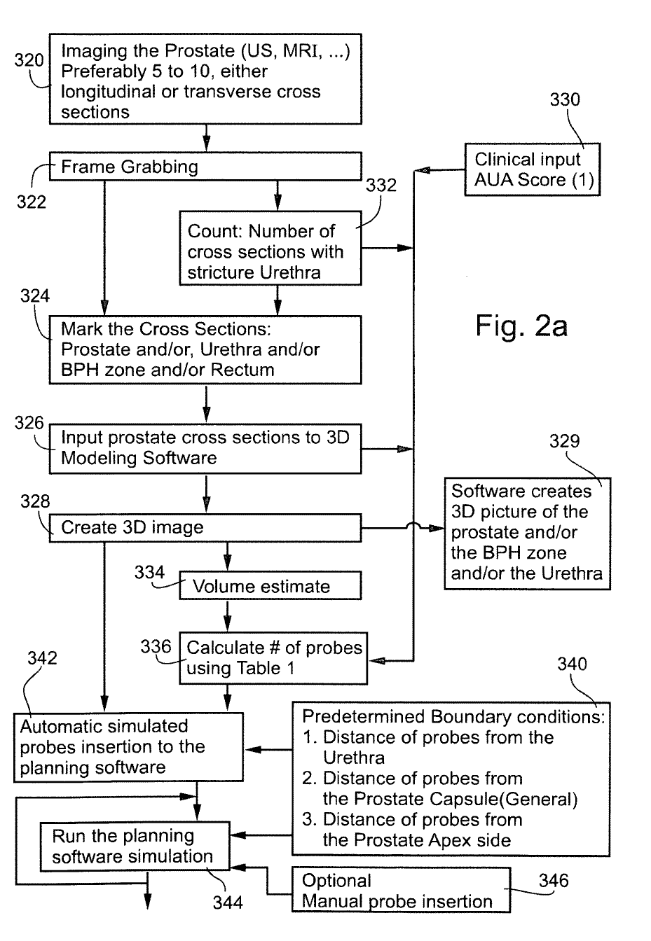

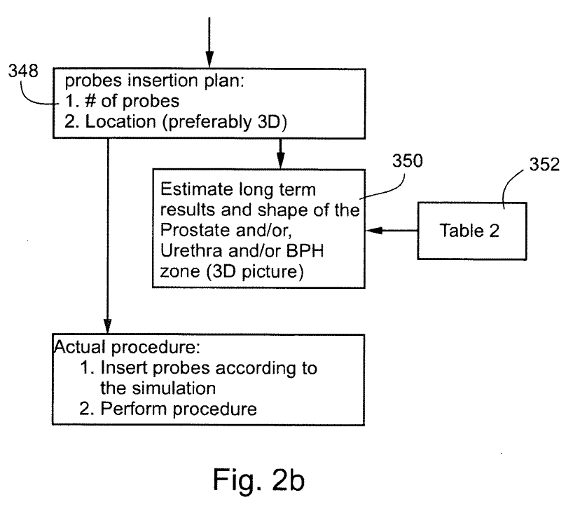

Reference is now made to FIGS. 2a and 2b, presenting a flow chart showing a method for automatically generating a recommendation relating to a cryoablation procedure, utilizing the information of Table 2, or similar information, according to methods of prior art. In the specific example of FIGS. 2a-2b, the generated recommendation is relevant to cryoablation of tissues of a prostate for treatment of BPH.

At step 320 of FIG. 2a, first imaging modality 250 is used to create preparatory images, which are digitized at step 322 to become digitized preparatory images 254. In the example presented, images 254 are cross sections of a prostate such as those generated by a series of ultrasound scans taken at regularly intervals of progressive penetration into the body of a patient, as might be produced by the ultrasound equipment described with reference to FIGS. 8-10 hereinabove.

At optional step 324, an operator marks or otherwise indicates, with reference to images 254, locations of tissues to be cryoablated or to be protected, as explained hereinabove. At step 326 images 254 are input to three-dimensional modeler 256, which creates three-dimensional model 258 of the intervention site at step 328. Model 258, along with any operator-highlighted and classified regions of model 258, are displayed at step 329.

In a parallel process, raw materials for a recommendation are gathered. At step 330 clinical input in the form of an AUA score from a questionnaire of a patient's symptoms is input. At step 332 a count is made of the number of preparatory images 254 (cross-sections) of the urethra which show constriction to the urethra caused by pressure from the prostate tissue on the urethra. A count of cross-sections showing constriction is here taken as an indication of the length of a stricture. Determination of which cross-section images show signs of constriction may be made by an operator, or alternatively may be made by automated analysis of images 256, using image interpretation techniques well known in the art. At step 334, information available to three-dimension modeler 256 is used to automatically calculate the volume of the prostate.

At step 336, information assembled at steps 330, 332, and 334 is used in a table-lookup operation to retrieve a recommendation for the appropriate number of probes to be used to treat the imaged specific case of BPH.

At step 340, an operator optionally inputs specific boundary conditions which serve to limit recommendations by the system. Utilizing model 254 created at step 328, operator-specified boundary conditions from step 340, operator-specified identification of locations of specific tissues to be ablated or protected from step 324, and a calculated recommended number of probes from step 336, a recommendation for optimal positioning of a recommended number of probes may be made at step 342. Display of a recommended intervention is made at step 344.

Optionally, operator-specified placement of simulated cryoprobes may modify or replace the recommended intervention, at step 346.

Step 344 is optionally iterative. That is, an operator may repeatedly modify definitions of tissues, boundary conditions, or manual placement of simulated probes, until the operator is satisfied with the simulated results. As a part of step 344, activities of evaluator 300 may be evoked, so as to procure system feedback based on a simulated intervention. Step 344 is repeated so long as desired by an operator, and until the operator is satisfied with the results.

Referring now to FIG. 2b which is a continuation of the flowchart of FIG. 2a, at step 348 a final plan is optionally saved to a computer disk or other memory 270.

In optional step 350, details of the completed intervention plan can be used to estimate and display expected long-term results of the planned intervention, such as an expected future volume and shape of the prostate. Information from Table 2 or an equivalent is utilized for step 350, as indicated at step 352. It is noted that long-term volume of the prostate may also be treated as a boundary condition of an intervention, at step 340.

The example presented in FIGS. 2a and 2b refers specifically to a utilization of planning system 240 for treating a prostate for BPH. Similar utilizations may be contemplated, for treating other organs, or for treating other conditions of a prostate.

In treating BPH, a desired goal is a reduction in prostate volume so as to relieve pressure on the urethra of a patient, because pressure on the urethra from an enlarged prostate interferes with the process of urination. In treating BPH there is no need to destroy all of a selected volume, but rather simply to destroy some desired percentage of that volume.

In treating, for example, a prostate tumor suspected of malignancy, goals of the intervention are quite different. To avoid dangerous proliferation of malignant cells, it is desirable to ablate a defined volume in its entirety. In such a context, when it is necessity to destroy all tissues within a selected volume, the functionality of evaluator 300 of planning system 240 is particularly useful.

Evaluator 300 is able to calculate, for each arbitrarily selected small volume of model 258, the cumulative cooling effect of all cryoprobes in proximity to said selected small volume. Consequently evaluator 300 is able to make at least a theoretical determination of whether, for a given deployment of cryoprobes utilized under a given set of operating parameters, total destruction of malignant tissues within a selected volume is to be expected.

Planning system 240 can be used effectively to plan a dense arrays of cryoprobes. For example, a user might specify a particular density of an array of probes, then use evaluator 300 to evaluate a range of possible temperature and duration parameters to find an amount and duration of cooling which ensures that the specified array will indeed create a nearly-uniform cold field sufficient to destroy all target tissues. Alternatively, a user might specify a desired degree of cooling and use planning system 240 to recommend a required density of the cryoprobe array.

Thus, evaluator 300 and recommender 310 can be used to calculate placement and operational parameters of cryoprobes in a manner which guarantees a nearly-uniform cold field within a selected volume. If cryoprobes 266 are sufficiently small and placed sufficiently close together, cooling effects from a plurality of probes will influence each selected small volume within a target volume, and an amount of required cooling can be calculated which will ensure that all of the target volume is cooled down to a temperature ensuring total destruction of the target volume.

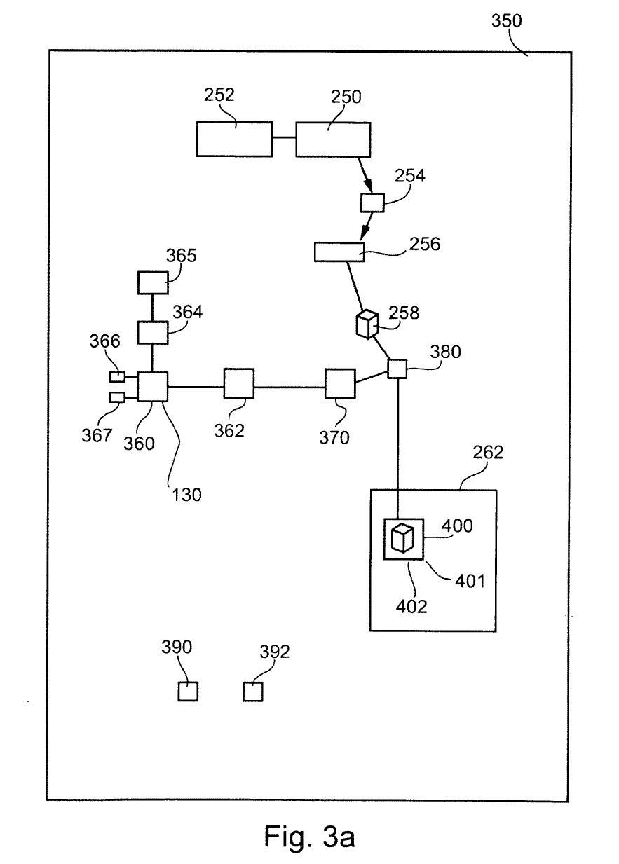

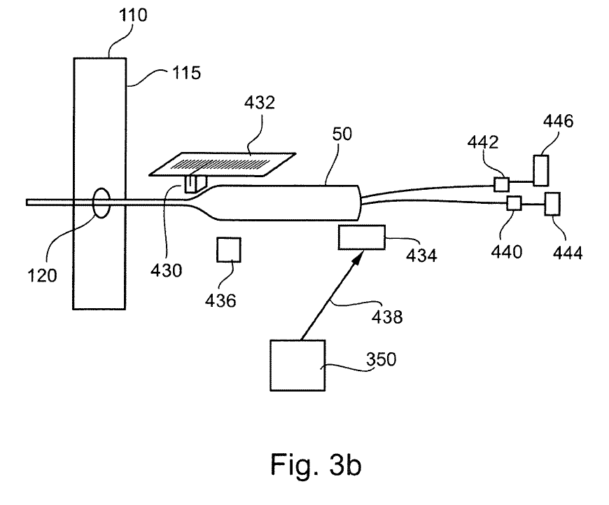

Reference is now made to FIG. 3a, which is a simplified block diagram of a surgical facilitation system for facilitating a cryosurgery ablation procedure, according to methods of prior art.

In a preferred embodiment, a surgical facilitation system 350 comprises a first imaging modality 250 and optional digitizer 252, for creating digitized preparatory images 254 of an intervention site, a first three-dimensional modeler 256 for creating a first three-dimensional model 258 of the intervention site based on digitized preparatory images 254, a second imaging modality 360 with optional second digitizer 362 for creating a digitized real-time image 370 of at least a portion of the intervention site during a cryosurgery procedure, and an images integrator 380 for integrating information from three-dimensional model 258 of the site and from real-time image 370 of the site in a common coordinate system 390, thereby producing an integrated image 400 displayable by a display 260. Integrated image 400 may be a two dimensional image 401 created by abstracting information from a relevant plane of first three dimensional model 258 for combining with a real-time image 370 representing a view of that plane of that portion of the site in real-time. Alternatively, a set of real-time images 370 may be used by a second three dimensional modeler 375 to create a second three dimensional model 402, enabling images integrator 380 to express first three dimensional model 258 and second three dimensional model 402 in common coordinate system 390, preferably a Cartesian coordinate system, thereby combining both images into integrated image 400.

Various strategies may be used to facilitate combining of model 258 (based on preparatory images 254) with real-time images 370 (or model 402 based thereupon) by images integrator 380. Processes of scaling of images to a same scale, and of projection of a ‘slice’ of a three dimensional image to a chosen plane, are all well known in the art. Basic techniques for feature analysis of images are also well known, and can deal with problems of fine alignment of images from two sources, once common features or common directions have been identified in both images. Techniques useful for facilitating aligning of both images by images integrator 380 include: (a) identification of common features in both images by an operator, for example by identifying landmark features such as points of entrance of a urethra into, and points of exit of a urethra from, a prostate, (b) identification of constant basic directions, such as by assuring that a patient is in a similar position (e.g., on his back) during both preparatory imaging and real-time imaging, (c) operator-guided matching, through use of interface 264, of a first set of images, (d) use of proprioceptive tools for imaging, that is, tools capable of reporting, either mechanically or electronically using an electronic sensor 364 and digital reporting mechanism 365, their own positions and movements, and (e) using a same body of imaging equipment to effect both preparatory imaging, producing preparatory images 254, and real-time imaging during a cryosurgery procedure, producing real-time images 370. For example, using ultrasound probe 130 of FIG. 3a both for preparatory imaging and for real-time imaging, and assuring that the patient is in a standard position during both imaging procedures, greatly facilitates the task of images integrator 380. Equipping ultrasound probe 130 with stabilizer 366 and controlling its movements with stepper motor 367, as shown in FIG. 3a, yet further simplifies the task of images integrator 380.

It will be appreciated that the described system can benefit from position tracking of various components thereof so as to assist either in modeling and/or in actually controlling a cryoablation procedure. Position tracking systems per se are well known in the art and may use any one of a plurality of approaches for the determination of position in a two- or three-dimensional space as is defined by a system-of-coordinates in two, three and up to six degrees-of-freedom. Some position tracking systems employ movable physical connections and appropriate movement monitoring devices (e.g., potentiometers) to keep track of positional changes. Thus, such systems, once zeroed, keep track of position changes to thereby determine actual positions at all times. One example for such a position tracking system is an articulated arm. Other position tracking systems can be attached directly to an object in order to monitor its position in space. An example of such a position tracking system is an assortment of three triaxially (e.g., co-orthogonally) oriented accelerometers which may be used to monitor the positional changes of the object with respect to a space. A pair of such assortments can be used to determine the position of the object in six-degrees of freedom.

Other position tracking systems re-determine a position irrespective of previous positions, to keep track of positional changes. Such systems typically employ an array of receivers/transmitters which are spread in known positions in a three-dimensional space and transmitter(s)/receiver(s), respectively, which are in physical connection with the object whose position being monitored. Time based triangulation and/or phase shift triangulation are used in such cases to periodically determine the position of the monitored object. Examples of such a position tracking systems employed in a variety of contexts using acoustic (e.g., ultrasound) electromagnetic radiation (e.g., infrared, radio frequency) or magnetic field and optical decoding are disclosed in, for example, U.S. Pat. Nos. 5,412,619; 6,083,170; 6,063,022; 5,954,665; 5,840,025; 5,718,241; 5,713,946; 5,694,945; 5,568,809; 5,546,951; 5,480,422 and 5,391,199, which are incorporated by reference as if fully set forth herein.

Position tracking of any of the imaging modalities described herein and/or other system components, such as the cryoprobes themselves, and/or the patient, can be employed to facilitate implementation of the present invention.

In a preferred embodiment, surgical facilitation system 350 further comprises all functional units of planning system 240 as described hereinabove. That is, facilitation system 350 optionally comprises simulator 260 having user interface 264 with highlighter 280, each having parts, functions and capabilities as ascribed to them hereinabove with reference to FIG. 1 and elsewhere. In particular, system 350 includes the above-described interface useable by an operator to specify placements and operational parameters of simulated cryoprobes 266, and to specify tissues to be cryoablated or to be protected during cryoablation.

Similarly, facilitation system 350 further optionally comprises memory 270, predictor 290, evaluator 300, and recommender 310, each having parts, functions and capabilities as ascribed to them hereinabove with reference to FIG. 1 and elsewhere.

Thus, in a preferred embodiment of the described system, facilitation system 350 is able to undertake all activities described hereinabove with respect to planning system 240. In addition, facilitation system 350 is able to provide a variety of additional services in displaying and evaluating at least one real-time image 370, and is further able to compare real-time images 370 to three dimensional model 258, and also to compare information from real-time images 370 to stored information such as that identifying operator-specified tissues to be cryoablated or to be protected, as is explained more fully hereinbelow.

In a preferred embodiment, either first imaging modality 250 and/or second imaging modality 360 may each independently be a magnetic resonance imaging system (MRI), an ultrasound imaging system, a computerized tomography imaging system (CT), some combination of these systems, or some similar system able to produce images of the internal tissues and structures of the body of a patient, yet in the case of second imaging modality 360, ultrasound and MRI imaging are more typically used, as being more conveniently combined with cryosurgery processes.

Facilitation system 350 further comprises a first comparator 390, for comparing first three-dimensional model 248 with real-time image 370, particularly to discern differences between both images. Such differences constitute differences between a status of a planned intervention and a status of an actual intervention in real-time. Tools, such as cryoprobes, tissues, such as a urethra, and ice-balls formed during cryoablation, all figure as elements in three dimensional model 258, and all may be visualized using second imaging modality 360. Thus, their expected positions, sizes, orientations, and behaviors may be compared to their actual real-time positions, sizes, orientations and behaviors during cryoablation, by comparator 390.

Differences thereby revealed, and information concerning such differences, can be of vital importance to an operator in guiding his actions during an intervention, particularly if the operator deviates from a planned intervention without being aware of doing so. A representation of the revealed differences may be displayed by displayer 262 and highlighted for greater visibility. A feedback mechanism 392, for example an auditory feedback mechanism, may be used to draw attention of an operator to serious discrepancies between a planned and an actual intervention.

Similarly, comparator 390 can be used to compare status of objects visible in real-time images 370 with stored information about operator-specified tissues to be cryoablated. Comparator 390 can thus provide information about, and displayer 262 can display, situations in which tissues intended to be cryoablated are in fact not effectively being cryoablated by a procedure. Similarly, comparator 390 can be used to check status of objects visible in real-time images 370, relating them to stored information about operator-specified tissues which are to be protected during cryoablation. In the case of discrepancies between an actual situation and an operator-specified desirable situation, display 262 and feedback mechanism 392 can warn an operator when a procedure seems to be endangering such tissues.

The capabilities of facilitation system 350 may extend yet further, to direct guidance to an operator in the manipulation of cryoablation tools, and even to partial or complete control of such tools during a phase of a cryoablation intervention.

Reference is now made to FIG. 3b, which is a schematic diagram of mechanisms for control of cryosurgical tools by a surgical facilitation system, according to methods of prior art.

A cryosurgical probe 50 is shown passing through an aperture 120 in a guiding element 115 which is realized in this example as a plate 110. Aperture 120 is for limiting sideways movement of probe 50, which is however free to move forward and backwards towards and away from a cryoablation site in a patient. In prior art methods such as that of Schatzberger discussed in the background section hereinabove, such movement is conceived as under sole and exclusive control of an operator who advances and retracts probe 50 manually.

As has been noted above, the simulation, evaluation, and recommendation capacities of planning system 240 and facilitation system 350, based on preparatory images 254 and three dimensional model 258, allow system 350 to calculate a recommended maximum and minimum depth for at which each cryoprobe 50 is to be used for cryoablation. Further, a cryoablation plan manually entered by an operator may also determine a maximum and minimum depth at which each cryoprobe 50 is to be used for cryoablation.

In a simple implementation of mechanical control based on information from planning system 240 or facilitation system 350, planned maximum and minimum depths generated by those systems are communicated to an operator who adjusts a mechanical blocking element 430 according to a graduated distance scale 432, in a manner which limits forward or backward movement of probe 50 so as to prevent an operator from unintentionally and unknowingly advancing or retracting probe 50 beyond limits of movement planned for probe 50. Such an arrangement guides and aids an operator in use and control of probe 50 for effecting cryoablation according to a plan.

In a somewhat more sophisticated implementation, control signals 438 from system 350 activate a stepper motor 434 to directly control movement of probe 50. Thus, under control of system 350 and according to a planned, simulated, examined and theoretically tested procedure, stepper motor 434 can advance probe 50 to a planned depth for performing cryoablation. System 350 can also send temperature control signals to heating gas valve 440 and cooling gas valve 442, thereby controlling a flow of heating gas from heating gas reservoir 444 and a flow of cooling gas from cooling gas reservoir 446. Thus, under control of an intervention plan and utilizing mechanisms presented herein, system 350 is able to directly control some or all of a cryoablation intervention. Thus, in a typical portion of a cryoablation procedure, stepper 434 advances probe 50 a planned distance, cooling gas valve 442 opens to allow passage of a gas which cools probe 50 to cryoablation temperatures and maintains those temperatures for a planned length of time, then cooling valve 442 closes to halt cooling. Optionally, heating gas valve 440 then opens to allow passage of a gas which heats probe 50 so as to melt tissues in contact with probe 50, thereby restoring to it freedom of motion, whereupon stepper motor 434 can further advance or retract probe 50 to a new cryoablation position, at which new position system 350 can optionally repeat this cryoablation process.

To ensure accuracy, movement of cryoprobe 50 may be monitored by a movement sensor 436. Moreover, all the facilities of system 350 previously described, for comparing real-time positions of objects with planned positions of those objects, can be brought to bear, to monitor this independently controlled cryoablation process.

Attention is now drawn to FIG. 4, which is a simplified schematic of a system 100 for planning and monitoring a probe-based surgical procedure such as a cryoablation, according to an embodiment of the present invention.

System 100 may be used to:
acquire one or more images image of a neighborhood of an lesion to be treated, typically utilizing one or more imaging modalities such as ultrasound, CT, MRI, x-ray, or other;
optionally receive information from a user relating to that image(s), in particular information identifying and localizing organs to be treated, information identifying and localizing organs or structures to be protected from damage (which information is referred to hereafter as the “desired outcomes”);
optionally integrate additional information from imaging modalities such as PET, fMRI and Nuclear Medical imaging (NM) and/or from non-imaging sources such as biopsy results, which information indicates probability of malignancy and/or desirability of tissue destruction in specific locations;
optionally receive from user or from a recorded information source a set of a commands and constraints relating to a cryotherapy operation to be planned and/or executed, (e.g. type and number of cryoprobes to be used, desired temperature profiles, optimization constraints, etc.)
plan a cryoablation procedure based on the received information. Planning may involve estimating outcomes of user-input commands and/or recommending probe placements and/or probe operational parameters such as time and intensity of cooling, pull-back protocols etc.;
optionally simulate (i.e. calculate) expected results of the planned cryoablation procedure, optionally displaying calculated results in a variety of formats, preferably including display of two-dimensional maps or three-dimensional models of the body region being treated, showing temperature isotherms and/or zones of probabilities of tissue destruction, preferably highlighting relationships of similarity or dissimilarity between these predicted outcomes and the desired outcomes;
optionally, use automatic means to insert cryoprobes into a patient, and/or assist a surgeon in inserting cryoprobes, and/or monitor insertion of cryoprobes and provide feedback to a surgeon regarding his insertions as related to the planned insertions;
acquire (from imaging modalities optionally annotated by user) information concerning actual positions of inserted cryoprobes after insertion is completed;
optionally, plan a cryoablation procedure based on actual detected cryoprobe positions;
perform and/or monitor performance of cryoablation procedure as planned while displaying process to user and accepting user override commands, optionally providing feedback and/or controlling procedure based on similarities and differences between planned and actual outcomes and/or between actual outcomes and desired outcomes.

FIG. 4 presents an exemplary component, here generally designated as system 100, which may be comprised in some embodiments of the present invention.

System 100 comprises a thermal ablation planning unit 136 having a display 138 and input device 139. Planning unit 136 is preferably a computer such as a PC or a laptop computer. Display 138 is preferably a flat panel graphic display such as LCD, but may be a CRT or plasma display, a stereoscopic display device, or other graphic display. Input device 139 preferably comprises a pointing device such as a mouse and may also comprise a keyboard. Optionally, input device 139 comprises a microphone and voiced recognition software for receiving voice commands and/or for recording voice comments. A plurality of input devices may be used.

An ultrasound control unit 122, connected to an internal ultrasound probe 124 is used for acquiring ultrasonic signals enabling to construct ultrasonic images of the tissue under treatment. For example, in treatment of the prostate ultrasound probe 124 is preferably a rectal probe inserted into the patient's rectal cavity, whereas in treatment of the uterus or its vicinity ultrasound probe 124 is preferably a vaginal probe inserted into the patient's vaginal cavity. Alternatively, ultrasound probe 124 may be an internal probe designed for insertion into any other natural or surgically made body cavity. As taught by Schatzberger, ultrasound probe 124 may be attached or otherwise physically related to a probe insertion guide (template) used for guiding insertion of therapeutic probes into a patient. A fixed physical relationship (or other known positional relationship) between probe and template simplifies registration of images provided by probe 124 with known locations of inserted therapeutic probes.

Optionally, internal ultrasound probe 124 may be connected to a motorized probe positioner 156 which is operable to control movement (e.g. advancement/retraction and/or rotation) of internal ultrasound probe 124 into and within the body cavity. Utilizing probe positioner 156 can facilitate acquiring a plurality of images having know spatial relationships one to another, from which a three-dimensional model of the organ or volume to be treated may be formed, as discussed above. In a currently preferred embodiment positioner 156 is used to capture a series of ultrasound images representing 5 mm steps from one image to the next. Each image is then used as a “slice” of a “multi-slice” 3-D image, useable to construct a three-dimensional model of the target organ and its neighborhood. Such a model and/or other form of 3-D image may be stored, used, and manipulated by planning unit 136 and displayed on display 138, optionally in stereoscopic format. Thus, display 138 can be used to display either a series of two-dimensional views or a three-dimensional view of the surgical target area, which views can be used for target analysis and planning. In a further preferred embodiment, a mechanically or electronically steerable ultrasonic probe having multi-dimensional freedom of motion/viewing-angle may be used.

In place of motorized probe motion mechanism 156, a manual probe motion mechanism 157 may be used, by means of which an operator manually manipulates probe 124. Manual mechanism 157 preferably is either designed to move probe 124 by known distance steps, or else comprises a position sensor operable to report probe position, facilitating registration of individual images in a three-dimensional context, enabling 3-D modeling as discussed above.

An external ultrasound probe 126 may be used together with, or instead of, ultrasound probe 124. In a preferred embodiment external ultrasound probe 126 is an abdominal probe. Ultrasound probe 126 may be used from a fixed position, or may be equipped with a position sensor 121. An example of such a sensor is the electromagnetic location sensor CARTO™ XP EP sold by Biosense Webster (Israel) Ltd, Tirat Carmel, ISRAEL, which may be seen at www.biosensewebster.com. Other similar sensors are well known in the art. Optional location sensor 121 provides information on the position and direction from which image views are taken, thereby providing information regarding the spatial relationships between objects visible in disparate views. Such information enables to register ultrasonic images taken by ultrasound probes 124 and 126 within a common fixed Cartesian coordinate system.

Use of a common coordinate system enables, for example, comparing of actual visible ice-ball location and size to planned ice-ball location and size. A common coordinate system is particularly important in cases where ultrasound probe 126 is moved during monitoring, and in cases where images taken for planning purposes are taken from a different viewing point from that used during surgery, or are made by a different imaging device.

Simultaneous use of both external and internal ultrasound probes, or for that matter simultaneous or coordinated use of two or more separated ultrasound probes viewing a same volume from different directions, and reconstitution of a composite image or three-dimensional model based on information supplied by images from both sources, constitutes, in the field of cryosurgery, a significant advance over methods of prior art. It is in the nature of cryosurgery that frozen tissue is opaque to normal ultrasound, consequently viewing from a single perspective cannot provide full information on the condition of a volume undergoing cryoablation. Thus an ultrasound probe inserted in a rectum, as is standard methodology in prostate cryosurgery, cannot reveal with clarity the state of tissues and the position of frozen tissues on the side of the prostate opposite the rectum.

System 100 overcomes this limitation by providing for use during surgery an ultrasound system comprising first and second ultrasound probes, each probe preferably associated with a position reporter operable to report its position and orientation, an image registration system operable to register information gleaned from operation both first and second probes in a common virtual space, and an image display system operable to display an image of a portion of that common virtual space, which image comprises information gleaned from both first and second probes. Position reporters operable to report positions of the probes may be position sensors 121 and 155, or may be functions of a command mechanism such as probe positioner 156 serving to control automated movement of one or both of the probes, or may even be a user interface used by an operator to manual report fixed or moving positions of probes manually controlled by an operator.

According to a preferred method of use, an operator positions a rectal ultrasound probe 126 in a rectum of a patient, and uses that probe to generate a first ultrasound image of a prostate, positioning an abdominal ultrasound probe 126 on the patient's abdomen directed towards the prostate to generate a second ultrasound image of said prostate, and then either simultaneously or alternatively displays the first and second images, providing the user with simultaneous or near-simultaneous views of the prostate from two different perspectives. An ultrasound timing coordinator 123 may be utilized to coordinate rapid alternation of functioning of probes 124 and 126, so as to provide nearly continuous readings from each probe, yet avoid acoustic signal interference between the probes. If rapid alternation of functioning of probes 124 and 126 is used, controller 122 can of course be programmed to avoid flickering of captured images by prolonging display of captured images when appropriate.

In a further preferred embodiment, system 100 uses image registration software to relate information gleaned from the first and second images to a common coordinate system, and displays a composite image of the prostate, the composite image comprising information gleaned from both first and second images. The described method thus produces images providing more complete information than would be provided by either the first or second images alone. This is particularly important in the case of cryosurgery of a prostate, since during cryoablation the large iceball(s) produced by the ablation process prevent ultrasound viewing of the side of the organ which is distant from the ultrasound probe. It is to be noted, however, that whereas this embodiment has been described in the exemplary context of cryoablation of a prostate, the invention is useful in treatment of various organs other than the prostate, and in clinical contexts other than cryoablation.

To aid in registration of images from both probes in a common coordinate system, in a preferred embodiment one or both of probes 124 and 126 are physically connected, optionally through a motorized or manual probe motion mechanism 156 (for probe 124) and/or 129 (for probe 126), to a common physical reference frame 153. Alternatively, some portion of the described mechanisms may be physically connected (e.g. a frame connected to an external ultrasound and also connected to a patient's bed) while other portions rely on position sensors or commandable servomotors to create a known relationship of components (such as probes 126 and 126) to a patient and to each other, that spatial interrelation system being referred to herein as frame 158. Thus a physically connected portion of frame 158 may be connected to a bed, or to a patient, and physically unconnected portions (e.g. an abdominal ultrasound) may be maintained in a known positional relationship to fixed portions of frame 158 by means of position sensors, stepper motors, measuring scales visible to a surgeon, etc.

One or more probe insertion aids 150 may be connected to reference frame 158 and be used for guiding probes cryoprobes (probes 135 and 135′ are seen in this figure) sensor probes, ultrasound probes and other probes to desired locations within the patient's body. Insertion aid 150 may for example be a cryoprobe insertion template similar to that taught by Schatzberger, as citied in the background section hereinabove.

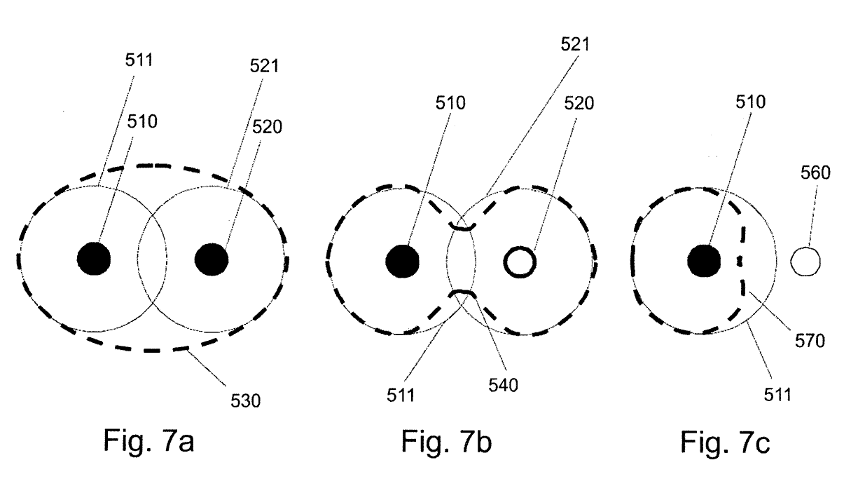

Cryoprobes probes 135 are connected via hoses 133 (two such hoses: 133 and 133′ are seen in this figure) to a cryogen control unit 134 provided to control supply of cryogen to cryoprobes 135, and thereby to control cooling and optionally heating of cryoprobes 135. If cryoprobes 135 are a Joule-Thomson cryoprobes, cryogen control unit 134 will be a controller operable to control supply of high-pressure cooling gas and optionally high-pressure heating gas. Cryogen control unit 134 supplies cryogen through hoses 133 to cryoprobes 135, where it traverses the shaft of cryoprobe 135 and is delivered to operating tips of cryoprobes 135, which tips are cooled by expansion of the cryogen (in the case of a Joule-Thomson cryoprobe) or by evaporation of the cryogen (in the case of an evaporative cryoprobe). Cryogen control unit 134 may be controlled by thermal ablation control unit 136, or alternatively may be manually controlled a user. In a preferred embodiment of system 100 discussed in detail hereinbelow with respect to a pattern of cryoprobe used presented in FIGS. 7a-7c, probes 135 are each operable both to heat and to cool, and cryogen control unit 134 is operable to individually supply to each probe 135 either a heating gas or a cooling gas.

Optionally, one or more thermal sensing probes 165 may be inserted in the vicinity of the treated organ, each thermal sensing probe 165 comprising one or more thermal sensors. One or preferably a plurality of thermal probes 165 may be connected a thermal interface box 164 using cable or wireless communication links. Signals indicative of temperature readings at probes 165 are transferred to planning unit 136 via thermal interface box 164.

System 100 may comprise one or more additional imaging apparatus 160, such as an x-ray imager, digital or film based camera, or fluoroscope. Additional imaging apparatus 160 are preferably connected to planning unit 136, providing additional sources of images of the treatment locus within the body of the patient, which images are used during planning and ablation phases of treatment. Optionally, additional apparatus 160 may be located remotely, images acquired thereby being electronically transferred to planning system 136 via communication link, or stored on removable memory media for subsequent uploading into planning system 136. Apparatus 160 might, for example, be an MRI at a remote site, used to create pre-operative images of the patient. CT cameras and functional imaging devices such as nuclear gamma camera acquiring planar images or Single Photon Emission Tomographic (SPECT) images or Positron Emission Tomograph (PET) are additional examples of additional imaging apparatus 160.

In preferred embodiments, thermal sensing probes 165 and ablation probes 135 comprise one or more echogenic surfaces, which surfaces aid in making such probes easily visible under ultrasonic imaging. Additionally or alternatively, these probes may comprise X-ray markers such as dense or opaque structures which aid in making such probes visible under x-ray imaging. It is noted that the location of a probe or other object in three-dimensional space may be deduced from two or more non-coaxial x-ray images.

In a preferred embodiment probes 135 have a shafts which comprises markings 137 visible under ultrasound or markings 132 visible under x-ray imaging (e.g. fluoroscope), which markings show visible measurements of shaft distances from distal operating tips of the probes, making it possible for an observer seeing probes 135 under an appropriate imaging modality to accurately measure the position of an operating distal tip of a probe 135 even when probe 135 is operated in cooling and its distal operating tip is not directly visible under the imaging modality because it is encased in an iceball opaque to the modality (as iceballs are opaque, for example, to ultrasound). Thus an observer of a visible portion of a shaft of such a probe 135 can calculate, based on markings 132 or 137, the position of the probe's invisible distal operating tip.

In an additional preferred embodiment system 100 comprises a vibrator 131 attachable to a probe 135 (or any other therapeutic probe of system 100). Vibrator 131 is operable to impart a high-frequency vibration to the probe 135 to which it is attached, while that probe is inserted in a patient, and even while the probe is operated in cooling. According to this embodiment ultrasound probes systems 126/126 include a Doppler detector 128 operable to detect a vibrating probe 128, by detecting Doppler variations in ultrasound echoes reflecting from the vibrating probe. According to this embodiment display 138 is operable to display the received ultrasound image while highlighting, within the image, echoes having a detected Doppler variation. Doppler detection of inserted probes can be particularly useful as an aid to registration of images in a common coordinate system. For example, two ultrasound images, one created by ultrasound probe 124 and another created by ultrasound probe 126, may easily be related to a common spatial coordinate system if one or preferably several inserted probes can be unambiguously identified in both images. Imparting a vibrational frequency to an inserted probe and utilizing Doppler detection to detect that probe provides an unambiguous means for automatically detecting that same probe in both images. Imparting different vibrational frequencies to a plurality of probes provides an unambiguous means for automatically detecting each of that set of vibrating probes in both images, making image registration relatively easy to accomplish algorithmically.

It is noted that functions ascribed herein to specific functional modules may be executed by other included functional modules without thereby altering essential aspects of the invention. For example, functions ascribed to interface box 164 or ultrasonic control unit 122 may be integrated into planning unit 136. Functions ascribed to planning unit 136 (e.g., display of ultrasound images) may be provided by a dedicated display associated with ultrasonic control unit 122.

In some embodiments, ultrasonic control unit 122 is a commercially available ultrasonic system equipped with matching ultrasonic probes and may comprise display, input devices, printer and visual output device. In these embodiments, planning unit 136 interfaces with ultrasonic control unit 122 at least to the extent of receiving from unit 122 ultrasonic images acquired by the ultrasonic system. Optionally, planning unit 136 may also receive from and transmit to ultrasonic control unit 122 signals indicative of various performance parameters, such as image zoom for example.

It should be noted that thermal ablation other than cryoablation may be performed using system 100 according to the current invention by replacing cryoprobes 135 with thermal probes.

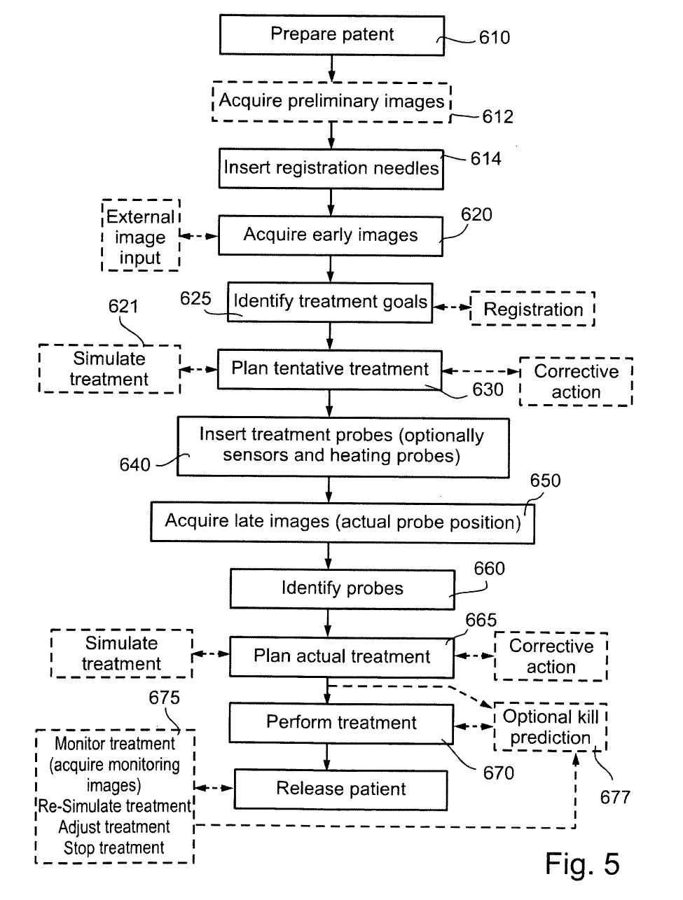

Attention is now drawn to FIG. 5, which is a simplified flowchart of a method for planning and control of a surgical intervention, according to an embodiment of the present invention. FIG. 5 details a procedure whereby system 100 acquires a first set of images of a patient (which set of images is referred to herein as “early images”), facilitates user input characterizing which tissues are to be ablated and which preserved, optionally receives user definitions of a cryoprobe setup (a set of cryoprobe insertion positions and cryoprobe operating parameters), calculates a predicted outcome of use of the setup and displays that outcome to a user, optionally plans (i.e. recommends) a cryoprobe setup, inserts cryoprobes according to a user-defined setup plan or a system-recommended setup plan, or enables and optionally assists a user to so insert a set of cryoprobes, reacquires patient images after probe placement is complete, optionally updates probe location information and user-defined treatment goals (characterization of target and non-target tissues), plans a treatment or allows a user to do so, optionally calculates expected treatment outcomes, enables corrective action to change probe placements if necessary, performs treatment according to plan, while monitoring, comparing real-time situations to planned outcomes and to user-defined treatment goals, and optionally adjusts treatment parameters during treatment and stops treatment according to plan or when required in response to a detected difference between planned and real outcomes. These procedures will now be discussed in detail.

Step 610 comprises preparing a patient for a surgical intervention, including positioning him appropriately with respect to components of system 100. Patient preparation typically comprises sedation (general or local anesthesia), attaching both patient and mechanical components of frame 158 to a bed, thereby fixing spatial relationships of patient and frame 158 and preventing motion and loss of registration, and positioning plate 150 (and/or a servomechanical device for inserting cryoprobes) with respect to the patient.

Patient preparation may also include insertion of rectal ultrasound probe 124. In preferred embodiments, rectal ultrasound probe 124 may be accompanied by a rectal warming mechanism. At optional step 612 preliminary images may be taken, using ultrasound probe 124 or other imaging modalities.

Preliminary images taken at step 612 serve for (optional) insertion of one or more marking probes 127 at step 614. Marking probes (also referred to herein as “registration needles” may be inserted into the target organ or into other structures in the vicinity of the target organ. Marking probes 127 are probes which are visible under ultrasound or another imaging modality, are easily identified within at least some early image. Marking probes 127 are inserted in known positions with respect to frame 158. For example, marking probes 127 may be inserted in a known aperture of a probe-guide template such as is taught by Schatzberger. Marking probes 127, being echogenic, are visible in early images, and may therefore serve to enable and facilitate registration of early images with reference frame 158.

Marking probes 127 are characterized by their visibility under the imaging modality in use. Thus, they may be simple echogenic probes with no other function. Alternatively, marking probes 127 may be cryoprobes 133, thermal sensors, or other functional probes with echogenic features (or radio-opacity, or similar characteristics of visibility under the imaging modality in use. According to a recommended mode of use, a cryoprobe 133 is used as a marking probe 127, and that cryoprobe 133, after being inserted into a target, is briefly operated in cooling, causing tissues of the target to freeze and consequently to adhere to cryoprobe 133, fixing cryoprobe 133 into a position from which it cannot be dislodged during subsequent phases of treatment, and in particular during insertion of additional treatment probes into the target.

Marking probes 127 are preferably left inserted until after therapeutic treatment probes 133 have been inserted, to further aid registration of early images with late images, as described below.

Step 620 comprises acquiring what will be called herein “early images” of the patient directed towards the locus of the intended intervention and its immediate environment within the patient's body. Early images may comprise a plurality of Two Dimensional (2-D) images, and may be used to form a three dimensional image or three-dimensional model of the site, utilizing modeling techniques well known in the art. Early images may combine information from plurality of sources, including internal ultrasonic probe 124, external ultrasonic probe 126, various imaging apparatus 160.

In a preferred embodiment, early images are presented to a user as one or more two-dimensional image slices of a target site, such “slices” being acquired directly from an imaging modality or reconstructed from a 3-D model constructed from information provided by imaging modalities. Thus, one or more early image “slices” may serve as a basis for treatment planning, as described below.

At step 225, treatment goals are identified.

In preferred embodiments, early images are presented to a user, who annotates one or more of those images by identifying, on the image, anatomical boundaries such as boundaries of tissues to be ablated or tissues to be protected.

Optionally, user identification on early images of marking probes 127 and/or anatomical features visible in the images, may serve to enable or facilitate completion of processes of registration of ultrasound images, other pre-surgical or real-time images, probe insertion aid 150 (e.g. Schatzberger template), servomechanical probe aids, and various other aspects and features of system 100, with respect to common frame of reference 158.

Additionally, step 625, identification of treatment goals, comprises identifying, in the context of frame 158 and its common set of spatial coordinates, tissues which it is desired to destroy by cryoablation. In most cases it will also be necessary or desirable to identify tissues desired to be preserved from damage during the cryoablation process. In preferred embodiments, early images are presented to a user, who annotates one or more of those images by identifying, on the image, anatomical boundaries such as boundaries of tissues to be ablated or tissues to be protected. Optionally, some or all of the process of so characterizing tissues may be done algorithmically by image analysis, yet in a preferred procedure, algorithmic characterizations of tissues, if supplied, are presented to a user for his approval or amendment.

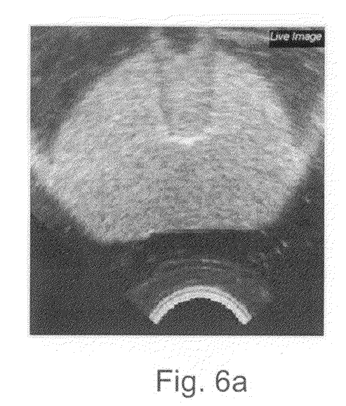

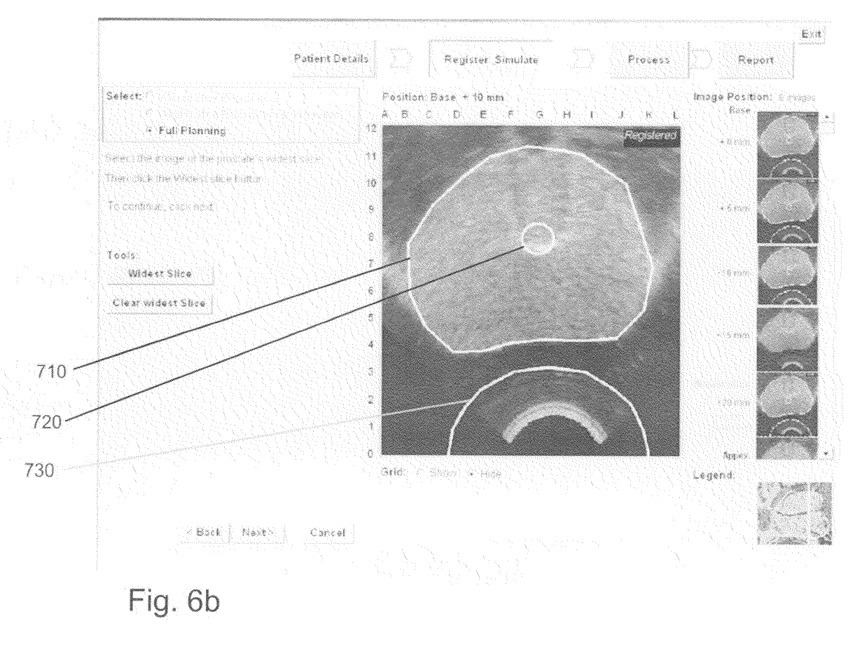

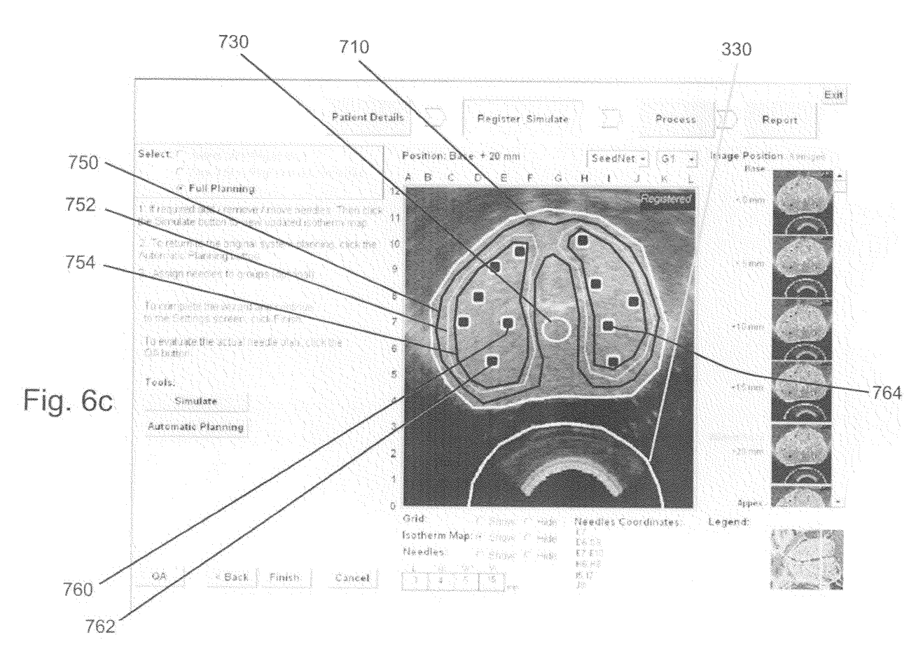

Attention is here drawn to FIGS. 6a and 6b, which illustrate this process. FIG. 6a is a raw ultrasound image of a prostate and its vicinity. FIG. 6b is an example of an annotated version of FIG. 6a, which version has been annotated according to an embodiment of the present invention. Markings on FIG. 6b indication positions of organ boundaries: as may be seen from the Figure, boundaries of a prostate 710, a urethra 720, and a rectal wall 730 are overlaid on this early image.

Identification and localization of boundaries of organs and lesions, and optionally identification and localization of other anatomical features useful for registering images or for other purposes, may be done by a user, by an automated system, or by a combination of an automated system supplying suggestions which are then accepted, rejected, or modified by a user. Image interpretation by algorithmic analysis is well known in the art. Success of any particular algorithmic approach will of course depend on the quality of the algorithm, the nature and quality of the early images being analyzed, and the degree of certainty of determination required by the clinical context. It seems probable that in many clinical contexts, particularly those in which questions of which tissues to kill are at issue, user supervision of the decision-making process, at least, will required for some time to come.

Accordingly, preferred embodiments of the present invention include features designed to facilitate tissue characterization by a user. As may be seen in FIG. 6b, an early image is preferably presented to a user in a familiar Windows-like graphical context, and the user is supplied with drawing tools of various sorts to facilitate his applying graphical marking directly to the presented image.

Thus, the step of establishing a virtual space map of a segment of a patient's body comprises the step presenting to a user at least one image that portion of the body, the image gleaned from an imaging modality, and receiving input from user, the input serving to identifying anatomical features present in that portion of the body marked by the user on the presented image.

To assist the user in this process, system 100 may utilize edge detection algorithms and curve-fitting algorithms to provide smoothed curve markers approximating detected edges of the image, as proposed boundaries. In some contexts system-proposed boundaries can be used as supplied, but in preferred embodiments users are invited to approve, disapprove, or modify boundaries proposed by the system.

Thus, for example, in treating a prostate, boundaries of the prostate will be identified in each of a sequence of ultrasonic ‘slice’ images. The plurality of 2-D boundaries thus input may be used as described above to create a 3-D model of the prostate. Structures internal to the organ, such as the urethra, and structures adjacent to it, such as the Neurovascular bundle or rectal wall, will be marked as well.

Geometric restrictions such as convexity smoothness of the resulted model may be imposed.

User marking of structure boundaries may be assisted by various facilitating features. For example, geometric restrictions such as convexity of the resulted model may be imposed. Initial ‘guesses’ by the system may be modified by ordinary graphics tools such as enlarge/reduce, shift, rotate, deform, etc. Optional initial guesses may be provided according to user-selectable preferences (e.g. “normal”, “enlarged”, “short”, “long”, etc.) Features may be provided enabling the user to point to a position on a boundary marker, hold down a mouse button and “pull” the boundary, where “spline” functions move the boundary marker under constraint of smoothness. Functions may be offered, enabling a user to mark several points and automatically generate a smooth curve connecting them. In other words, a variety of graphical manipulation options may be offered to simplify and otherwise facilitate the process of user marking of anatomical boundaries. Of course, once boundaries have been graphically marked, system software translates the graphical marks on screen images into coordinates in the virtual 3-D space of frame 158, for use in relating to and interpreting subsequently received images, optionally for use in controlling cryoprobe insertions, and optionally for evaluating and controlling ablation procedures.

In some embodiments, automatic image processing software determines the structure boundaries. For example, a fitting algorithm may use an initial guess and iteratively optimize the boundaries' shape to achieve best fit to the acquired image. Optionally the user may assist the software by choosing the initial guess or modifying it as described above to approximate the organ's shape before the fitting algorithm starts. Optionally, once the boundaries are determined, the user may accept or modify them or optionally re-acquire the image and re-start the process.

In preferred embodiments, an additional marking facilitation feature is supplied. A database of feature markers 111 (shown in FIG. 4) may be maintained within a memory 112 of a feature matching module 113, which feature markers are characterized according to measured characteristics of patient types or organ types, and by general characteristics. Feature-matching module 113 can then use inputted or discovered information characterizing a particular patient or organ to search database 111 for a feature matching marking likely to fit a feature of a particular patient and organ by virtue of known similarities between actual patient and searched database entry. The found feature marker can then be superimposed over the early image on a trail basis, be accepted or rejected by the user, or be moved or graphically modified by the user to enhance the ‘fit’ between marker and anatomical boundary visible to the user on the early image. In other words, system 100 assists a user to identify an anatomical feature by providing, superimposed on a early image on a trial basis, a feature marker derived from a collection of feature markers expected to resemble a anatomical features of that expected type (e.g. the anterior wall of a prostate), and by enabling the user to use the presented feature marker to mark an anatomical feature in the presented image. Preferably, the feature marker presented by the system is selected from collection 111 of feature markers according to similarities between physical or symptomatic characteristics of the patient and indexed characteristics describing feature markers of collection 111. In an optional version of this embodiment, feature marker collection 111 may derive from a collection of marked features of actual patients, and physical or symptomatic similarities between actual patient and historical patient may be used as a function of database selection.

To further facilitate user marking of anatomical features in images, system 100 may accept input from a user with respect to one early image, then reproduce that user input in a similar position on another early image, thereby enabling the user to identify an anatomical feature present in said late image by modifying the reproduced input with respect to the late image. Thus, anatomical boundaries marked on one ‘slice’ 2-D image of early images (e.g. an ultrasound slice image taken at a particular depth of penetration of an ultrasound probe 124) can be tentatively transferred to an image of another ‘slice’ 2-D image (e.g. an ultrasound slice image taken at another depth of penetration of ultrasound probe 124), there to be accepted, rejected, moved or modified by a user as described above.

In the case of marking a sequence of ‘slice’ images representing a sequence of ultrasound images taken at known distances one from another, interpolation between marked boundaries on two images may be used to propose approximate tentative boundaries on a third image between the two. Thus one might, for example, mark prostate boundaries on a first (e.g. most shallow ultrasound probe penetration) slice image showing a prostate, on a last (e.g. deepest ultrasound probe penetration) slice image showing the prostate, and on that slice image showing the broadest prostate image. Then, by matching a curve (in the depth dimension) to portions of marked boundaries on first, last and largest slices, good approximations of boundaries on intervening slices may be achieved. Each time a user approves or modifies a border on an intervening slice, interpolation of the other as-yet-unapproved slices may be updated using the collection of user-confirmed boundaries in the user-examined slices, resulting in progressively better and better fit between system-proposed boundaries and actual boundaries, and thereby facilitating user input of full organ boundary information.

Along with organ boundary marking, additional information may be supplied. For example, if in prostate surgery a urethral warming catheter or rectal warming device is to be used, an operator might input this fact to system 100, optionally specifying operating temperatures of these devices, which information would be used during various calculations to be described hereinbelow.

In prior-art systems treatment goals are designated in ‘black and white’ fashion, with any given tissue being designated as marked for destruction, marked for preservation, or unmarked. However, according to preferred embodiments of the present invention, tissues are characterized according to a graduated scale, which scale which extends from characterizing tissues as being highly desirable to be destroyed to characterizing tissues as being highly desirable to be preserved, with a plurality of optional gradations therebetween. User marking of image regions according to such a graduated scale may be accomplished utilizing standard graphics tools much as described above, with the addition of standard graphics tools for ‘painting’ (i.e. characterizing) large image areas. Gradations along a ‘desirability-of-destruction’ scale may be indicated by transparent color overlays or by any similar graphic means. Users are of course expected to input such information based on sources of generalized clinical knowledge (general clinical experience), patient-relevant information sources (biopsy results, clinical test results, etc.), clinical readings and interpretation of images, etc.

In addition to user-specified scoring or weighting on a ‘desirability of destruction’ scale, similar input may in some cases be gleaned from images derived from imaging modalities under automatic or semi-automatic analysis. For example, in a preferred embodiment weighted desirability-of-destruction scores for body regions may be generated automatically or semi-automatically as a function of image intensity of pixels of an image supplied by an imaging modality (e.g. tumor scintigraphy PET scans) wherein image pixel intensity is known to be correlated with probability of malignancy.

With reference again to the treatment process described by FIG. 5, in an optional step 621 which may be practiced before or after user identification of treatment goals, a user may choose to enter a “simulation mode” in which he inputs to system 100 his selection of locations for insertion of cryoprobes and user-defined parameters for operating those probes, the locations being defined with respect to a early image registered with frame 158. Planning unit 136 then uses this input information as input to a thermodynamic modeling system operable to simulate effects of the defined treatment over time, and to predict temperature outcomes throughout the treatment locus. Suitable simulation software is available commercially, for example from Noran Engineering Inc., of Westminster, Calif. (http://www.nenastran.com).

In preferred embodiments, users then view the simulated treatment outcomes. Treatment outcomes may be presented in the form of temperature isotherms imposed on early images, or graphed over time for selected user-designated positions, or may indeed be presented in the form of an animated ‘movie’ of treatment outcome situations showing isotherm progression over time in sequential images over all or part of a planned span of treatment. Such animated presentations can of course be run at a speed and temporal direction which is under user control. Users may also control the frame of reference of any of the displays mentioned above: since outcome displays derive information from calculated values in a three-dimensional model in virtual space, and may be displayed as still or animated images of a selected plane within that space, position and orientation of the plane to be displayed may also be put under user control. Additionally, using methods well known in the art, the four-dimensional information set (three physical dimensions plus time) may in fact be subject to user-controlled animated displays of any two of the four dimensions shown as a sequence of images of any selected two dimensions varying over a third dimension. Further additionally, using techniques well-known in the art of stereoscopic display, three-dimensional information varying over time can be displayed as a stereoscopic three-dimensional animation giving a viewer a true ‘in depth’ sensory experience of projected progression of the ablation process over time.

In a further preferred embodiment, an estimation function or table of estimated or observed clinical outcomes may be used to convert time and temperature information for each location into an estimate of tissue survival for that location, and these tissue survival estimates may be presented, for example in the form of shadings or transparent color variations imposed on early images showing expected tissue survival probabilities at selected user-designated times or at end of treatment, or, using tabular lookup methods described hereinabove with respect to prior art, show projected tissue survival percentages or probabilities at a future time. Data relating to survival of tissues of particular organic types under varying conditions of cooling over time are available in the clinical literature. Calculated survival percentages can be displayed with colors or pixel intensities or shadings of various sorts used to show projected tissue survival probabilities. Here too, an animated ‘movie’ rendition can dramatize expected treatment processes, for example relating projected tissue survival probabilities to expected iceball dimensions with respect to a given set of cryoprobe emplacements and operating parameters.

The simulation/prediction process described above may be undertaken iteratively, with the user amending his selection of probe locations, moving, adding or removing marked probe or probes.

It is noted that if step 621, simulation of treatment, is practiced after a user has identified treatment goals, then simulation 621 may further comprise a comparison of treatment goals with simulated outcomes. If weighted ‘desirability-of-destruction’ scores have been entered, an outcome display may use complementary graphics modes (e.g. color+symbol overlays) to display an image combining user-supplied tissue-preservation-desirability scores with a map of predicted tissue destruction probabilities according to a given set of designated cryoprobe positions and cryoprobe operating parameters. Alternatively and perhaps preferably, graphical or other types of feedback may be supplied to dramatize or emphasize particularly high or particularly low correlations between what a user has identified as a desirable profile of tissue destruction, and what a simulation has predicted as a profile of tissue destruction probably to be expected under a defined set of probe placements and probe operating parameters. Colors or light intensities or other graphic feedback devices may be used to display a fine-resolution map of the comparison of weighted ‘desirability-of-destruction’ scores with calculated probability of destruction scores, for example by tinting in green areas where goal status and predicted output status agree, tinting in red areas where goal status and predicted output status disagree, and using a range of colors between green and red to show intermediate degrees of status agreement.

Thus, temperature outcomes, minimum temperatures, maximum temperatures, tissue survival probabilities, user evaluations of tissue survival desirability, plots of survival desirability vs. survival probability, and various other calculable factors and combinations or comparisons of factors can be calculated and displayed, in stereoscopy or on user-selected two-dimensional planes, in still images and in animated temporal sequences, for any part of the images area and any part of the projected treatment period. In particular, in preferred embodiments the user can command a display of minimal or maximal temperatures at locations on a user-selected plane, can display expected percentage of tissue destruction at a selected treatment time on a user-selected plane, can use colors within an early (or late) image to express expected percentage of tissue destruction, or scores of desirability of tissue destruction, or a correlation between this two latter values, varying over time, at user-selected positions or on a user-selected plane. The user can display a graph of a tissue condition over time for a specific tissue location, or can produce a plot of tissue condition over time along a user-selected one-dimensional line.

Referring again to FIG. 5, at step 630 the user optionally requests, and planning unit 136 prepares, a tentative treatment plan to achieve the identified treatment goals of step 625. Optionally, the user inputs general parametric requirements and constraints, and may express preferences in terms of priority weights for use in comparisons of potential outcomes. Thus for example a given user may express a preference for speed of operation over minimization of costs as determined by number of needles or amount of cryogen expended, or may select or limit the number of probes to be used, or may specify that the treatment plan may or may not use probe pullback techniques requiring a thawing phase between freezing phases, or expressing a preference for symmetrical or unsymmetrical distributions of cryoprobes, and may specify acceptable levels of tissue destruction uncertainty (which levels will, of course, be radically different when treating a malignancy than when treating, say, BPH), may impose treatment length limitations relating, for example, to the desirability of reducing risks imposed by prolonged anesthesia, and so on. Even ‘non-medical’ constraints may be taken into account, such as cost differentials among treatments, optimization of surgeon time, and so on. In general, scores for all such aspects of treatment can be factored into a global score for each “treatment outcome”, with various factors being weighed according to a scale of relative importance preferably supplied as a default or according to a set of standard usage profiles, and further modifiable by a user.

These general conditions having been specified by a user, or default values or standard value sets being applied, planner 136 may generate a treatment plan. The treatment plan comprises a recommended set of cryoprobe insertion positions and operating parameters, and, generally speaking, may be calculated by a highly iterative process of creating a large number of tentative placement schemes using simple placement rules which serve primarily to avoid doing massive calculations on obviously useless configurations, by comparing calculated outcomes of cryoprobe placements to identify those with relatively high success rates, and then iterating through the identified placement combinations with varying probe operation parameter settings to identify the best outcomes, which are then presented to a user for approval.

The treatment plan selected by system 100 is then preferably presented to the user.

Attention is drawn to FIG. 6c, which presents the exemplary user-interface screen wherein aspects of a calculated treatment plan are presented to a user, according to an embodiment of the present invention. Predicted isotherm positions 750, 752, 754, etc., and recommended probe locations 760, 762, 764 may be easily seen in the Figure.

A system-selected treatment is preferably presented to the user together with a summary of predicted treatment outcomes and other characterizations of the plan. For example, in addition to presentation of the total plan score, the plan score may be contextually characterized in various ways. For example, the total score may be broken down into its components (partial scores as related to the various weighted criteria), may be presented in a manner showing availability or lack of availability of alternative plans with similar scores, may be presented in a normalized context enabling to compare that score to average scores of similar treatments (e.g. historical treatments, known to the system, of same organs of similar size), and so on. Optionally, rule-based characterizations may be provided, including plain-language interpretations such as, for example “Plan is within acceptable outcome range.”, or “No acceptable plan can be found if planning is restricted to the specified number of cryoprobes.”

Having been presented the system-recommended plan in its context, the user then accepts, rejects, or modifies the plan. The user may modify the plan by modifying the evaluation process (e.g. by modifying the evaluation weights given to various criteria (e.g. weight of cost of cryogen vs. weight of ‘cost’ of patient comfort). Alternatively, the user may simply manually input a new or changed cryoprobe insertion location or cryoprobe operating parameter, and re-run the evaluation simulation. Or further alternatively, the user may ask the system to present other configurations with scores close to that of the configuration first presented.

In step 640, cryoprobes and optionally monitoring probes are inserted into the tissue, preferably using the recommended locations from the tentative treatment plan. Preferably, a template 150 registered to reference frame 158 is used to guide the user to manually insert the probes. Alternatively, a semi-automatic apparatus may be used for insertion of probes into the tissue, wherein the user manually inserts the probes under guidance of probe positional sensors and feedback mechanisms. Further alternatively, a fully automatic apparatus such as robotic apparatus may be used for insertion of probes into the tissue.

In step 650, a new image or preferably a plurality of new images of the organ to be treated are acquired. Images created at this stage are referred to herein as “late images”.

Sources, methods of acquisition and methods of analysis of “late” images are similar to those of “early” images, and so will not be again presented in detail. The primary difference between late and early images lies in the fact that early images are created before a plurality of therapeutic probes and optional sensors and warmers have penetrated target tissues: late images are created after most or all therapeutic probes are inserted in the target area. It has been found that the process of inserting a plurality of therapeutic probes may move or displace or distort all or parts of an organ, which displacement risks rendering invalid calculations of probe positions and probe operating parameters which appeared optimal before probe insertion took place. Tissue resistance, probe flexibility, tolerances in guidance equipment, human error, and various other sources of insertion inaccuracies can cause actual location of inserted probes to depart significantly from those probes planned and intended locations. So, in preferred embodiments of the present invention, at step 660 late images acquired at step 650 may, if necessary, be examined algorithmically or manually, and inserted probes (and, if necessary, anatomical features) re-identified by users as required, using the methods of step 625 and other methods disclosed herein.

Once these late images are thus re-registered and actual positions of inserted probes and organ boundaries are re-identified, at step 665 a user is again preferably given opportunities to simulate treatment output under these newly defined conditions, to modify probe positions or probe operating parameters, and to request, receive, select and optionally modify system-selected treatment plans, and in general to engage in the same kinds of investigative and evaluative activities as were available in step 630, with the difference that simulations, planning runs and evaluations are now performed based on actual positions of organs and cryoprobes have been inserted and are no longer likely to further move nor likely to further cause movement or further distortion of body organs. Under these new conditions of real rather than hypothetical cryoprobe and organ placement, simulated treatment outcomes can be inspected to determine whether treatment goals will be adequately met, automated treatment planning may be optionally re-run if considered necessary or desirable, and if projected outcomes are not sufficiently successful under the new circumstances actual probes can be actually repositioned and the whole process repeated until a successful outcome is predicted.

In step 670, treatment is undertaken. Optionally, new images may continue to be acquired and treatment outcomes may continue to be evaluated throughout the ablation procedure, results may be displayed to a user to facilitate his processes of manual control of the operation by providing him with constantly updated status information and outcome predictions. Alternatively, some or all control of the process may be taken over by the evaluation software of planner 136, which can use the same evaluation procedures previously discussed to determine whether a dangerous departure from expected and/or desired tissue conditions exists or may be expected to exist, and to recognize when treatment goals have been fulfilled, shutting down ablation procedures in timely fashion when goals are about to be met. In particular, since ice-ball boundaries generally bear a known (if approximate) relationship to ablation volume boundaries, ice-ball boundaries, which are easily detected in ultrasound images, may be monitored and used for issuing alerts to users and/or for standard and/or emergency automated control of cooling temperatures, termination of cooling, etc. (The specific relationship between ice-ball boundaries and ablation volume boundaries will depend on the tissue being treated and other specifics of the treatment goals, such as the required degree of certainty of total ablation, etc.).

Attention is now drawn to FIG. 8, which is a simplified schematic of a probe insertion system, according to an embodiment of the present invention.

FIG. 8 presents a probe insertion system 2000. System 2000 is presented in FIGS. 8-15 and in the accompanying text in the form of an exemplary implementation, as a cryotherapy system 2000A. Hence much of the discussion hereinbelow refers to “cryoprobes”. It is to be understood, however, that references to cryoprobes and cryotherapy are intended to be exemplary and not limiting: the systems and methods presented hereinbelow are well adapted for insertion of therapeutic probes of many types, and references to “cryoprobes” should be understood to refer to cryoprobes as examples of therapeutic probes in general.

Thus, FIG. 8 presents a probe insertion system 2000 represented by an exemplary cryotherapy system 2000A comprising a plurality of therapeutic probes 1999 here presented as cryoprobes 2010 connected by cryogen supply tubes 2012 to a cryogen supply module 2014. Cryogen supply module 2014 is operable to supply cryogen for cooling probes 2010, and also operable to supply a heating gas such as helium or an electric current or other power source for heating probes 2010. System 2000 also comprises a probe insertion apparatus 2100 operable to sequentially insert probes 2010 into a patient 2001.

A presently preferred embodiment of system 2000 comprises four or more cryoprobes 2010.

In some embodiments system 2000 comprises a controller 2150, discussed in detail below.

Insertion apparatus 2100 comprises an immobilizer 2110 which serves to maintain apparatus 2100 in a fixed spatial relationship to a patient. Immobilizer 2110 may comprise straps, bands, clamps or other attaching means for attaching apparatus 2100 directly to a patient. Alternatively, immobilizer 2110 may comprise straps, bands, clamps or other attaching devices which can be used to attach both apparatus 2100 and a patient to an intermediary object 2224 such a platform or bed or operating table, thereby establishing a stabilized spatial relationship between patient and apparatus 2100. It is noted that immobilizer components may be present as components of external devices associated with use of system 2000. For example, clamps of an operating table may function as immobilizer 2110 by clamping both apparatus 2100 and a patient to that operating table.

In some embodiments of system 2000 immobilizer 2110 serves to attach apparatus 2100 to an ultrasound probe 2220, such as a rectal ultrasound probe or a vaginal ultrasound probe. Alternatively, immobilizer 2210 may attach apparatus 2100 to a framework 2222 serving to hold an ultrasound probe in a stabilized physical relationship with a patient, thereby establishing a known and stabilized physical relationship with a patient and enabling ultrasound probe 2220 to produce ultrasound images of portions of patient anatomy at known positions.

In some embodiments, system 2000 serves as probe insertion aid 150, discussed hereinabove, and immobilizer 2110 is, or attaches to, physical reference frame 153 and spatial interrelation frame 158, also discussed above.

Insertion apparatus 2100 further comprises a cryoprobe grasper 2120. Grasper 2120, discussed in detail hereinbelow, serves to grasp cryoprobes 2010. Grasper 2010 may also be designed to enable grasping of therapeutic probes without cooling capacity which may also be inserted and manipulated by system 2000, such as heating probes 2016 and thermal sensor probes 2018.

In some embodiments, some or all of probes 2010, 2016, 2018 and other probes useable in system 2000 comprise markings 2019 identifying probe-type of each probe. Probe-type may include designation of function (heating, cooling, thermal measuring), size, and other characterizations. Markings 2019 may be visible markings such as printed identifying names or numbers, bar codes or similar codes, coded bands, colors, or other visible markings, may be marking detectable by electronic means, such as magnetic dots detectable by magnetic detector, patterned light reflectors, radio frequency tags, or may be other visible or invisible markings detectable by detectors within system 2000.

System 2000 may also comprise one or more cryoprobe introducers 2017. Introducer 2017 comprises a hollow or a plurality of channels for containing a plurality of probes 2010 or other probes and introducing them into a patient in a common insertion from which they may be individually or collectively deployed. Gripper 2120 may be sized and configured to grip introducer(s) 2017, and system 2000 may be configured to insert and remove introducer(s) 2017 in a manner similar to that described herein for inserting and removing cryoprobes.

In some embodiments grasper 2120 comprises a probe-type detector 2122, operable to detect the probe-type of probe grasped by grasper 2120 or approached by grasper 2120. In some embodiments probe-type detector 2122 is an optical detector such as a bar-code reader or a camera accompanied by a controller with image-interpretation software operable to read words or to recognize colors, dots, bands or other visible symbols appearing on probes. In some embodiments probe-type detector 2122 is a magnetic detector or a detector of radio signals. Signals from probe-type detector 2122 enable controller 2150 or other components of system 2000 to determine whether or not a probe grasped by 2120 has been correctly selected. In some embodiments detector 2122 enables system 2000 to approach gripper 2120 to a plurality of probes and to select, from among them, a probe called for by a probe insertion plan. In some embodiments detector 2122 enables system 2000 to determine whether a probe placed in gripper 2120 by a user or other agent is in fact a correctly selected probe, by comparing a detected probe type to a probe type called for by a probe insertion plan.

Insertion apparatus 2100 further comprises a positioner 2120 for positioning grasper 2120 with respect to patient 2001, and optionally comprises an inserter 2140 operable to linearly advance a probe 2010 grasped by grasper 2020.

Controller 2150 comprises a command module 2152 operable to calculate and communicate to apparatus 2100 a command sequence 2154 commanding apparatus 2100 to insert a distal end (treatment tip) of a cryoprobe 2010 grasped by grasper 2120 into a defined locus 2011 within a body of said patient.

Controller 2150 may comprise a memory 2155 for holding definitions 2013 of a plurality of loci 2011 defined within the body of patient 2001, and command module 2152 may be operable to calculate and communicate to apparatus 2100 a plurality of command sequences 2154 each commanding apparatus 2100 to insert a distal end of a cryoprobe 2010 or other probe grasped by grasper 2120 into one of said plurality of loci 2011.

In some embodiments each command sequence 2154 comprises a set (i.e. zero or one or more) of positioner commands 2156 commanding positioner 2130 to move grasper 2120 towards one of loci 2011, and a set (i.e. zero or one or more) of inserter commands 2158 commanding inserter 2140 to advance a grasped cryoprobe 2010 towards that locus. In most contexts of usage each command sequence 2154 will comprise at least one positioner command 2156 and at least one inserter command 2158.

Some embodiments of system 2000 comprise a locus-defining module 2180 operable to define a plurality of loci 2011 based on at least one image received from an imaging modality 2218. Imaging modality 2118 may be ultrasound 2220 or any other imaging modality, such as an MRI, a fluoroscope, an x-ray machine, a CT, a PET scanner etc. In one example, imaging modality 2218 may be ultrasounds 124 and/or 126 discussed above, and locus-defining module 2180 may be, or communicate with, planning unit 136 discussed above.

Locus-defining module 2180 may comprise a user interface 2182 receiving user input defining a locus 2011. Locus-defining module 2180 may be designed to enable a user to define a locus 2011 with respect to an image 2219 received from imaging modality 2218. That is, interface 2182 may present to a user one or more images, such as ultrasound images of a body, the images being registered to apparatus 2100 in such a way that positions on the image can be related to locations within the patient's actual body, and the user may be enabled to mark or otherwise indicate on the provided image or images loci where he wishes to insert cryoprobes or other therapeutic probes.

Alternatively and additionally, module 2180 may be, or comprise, or communicate with, planning unit 136 or a similar unit operable to define loci for probe placement. As described hereinabove, module 2180 may receive user input defining anatomical structures recognizable in image(s) 2219, may receive user-defined treatment goals, characterizations of tissues to be protected and tissues to be destroyed including tissue characterizations rated on a graduated scale of desirability of destruction, and may undertake image analysis for purposes of so characterizing tissues, and may calculate loci for probe insertions based on this information and/or any other information. Module 2180 may thus use any of the features and functions described hereinabove with reference to FIGS. 1-6, and in particular, those features and functions relevant to definition of loci 2011.

In some embodiments, controller 2150 is programmed to manage various aspects of a cryosurgery operation.

In some embodiments controller 2150 is programmed to order a plurality of movement command sequences 2154 in a manner which enables insertion of a plurality of cryoprobes into a patient in such order that early-inserted cryoprobes 2010 do not impeded movement of positioner 2130 during insertion of later-inserted cryoprobes. In some embodiments controller 2150 is programmed to order a plurality of movement command sequences 2154 in a manner which minimizes tangling of cryogen supply tubes 2012 supplying cryogen to the various cryoprobes 2010. For both of these purposes, optimal command sequencing will depend on such factors as the particular embodiment of system 2000, positions of patient 2001 and of cryogen supply source 2014, etc., but for most purposes a simple sequencing algorithm such as “insert that uninserted cryoprobe which is closest to the bottom left-hand corner of the range of positioner 2140, then repeat” will suffice.

In some embodiments, controller 2150 is programmed to control heating and cooling of cryoprobes 2010 by sending commands to cryogen supply module 2014, causing module 2014 to supply cryogen for cooling selected cryoprobes 2010, and/or causing module 2014 to supply heating gas or electricity or another power source for heating selected probes 2010, each probe being heated or cooled as commanded by controller 2150.

In some embodiments controller 2150 is thus operable to command insertion of a plurality of cryoprobes at selected loci, to command cooling of those inserted probes, and optionally to command heating of those inserted probes to provoke melting of tissues adjacent to the probes and thereby facilitate extraction of the inserted probes.

System 2000 can also be used to remove inserted probes. In some embodiments controller 2150 is programmed to remember (in memory 2155 or elsewhere) positions of cryoprobes inserted into patient 2001, and comprises programming for calculating and communicating to positioner 2130 a command sequence directing positioner 2130 to position gripper 2120 at a cryoprobe 2010 (or other therapeutic probe) previously inserted by apparatus 2100. Thus, system 2000 can be caused to insert a first cryoprobe at a first locus, release that first cryoprobe, insert other cryoprobes at other loci, optionally cool some or all of the inserted cryoprobes and/or optionally heat some or all of the inserted cryoprobes, and then be commanded to return to that first cryoprobe and to grip it in gripper 2120.

In some embodiments of the invention, gripper 2120 is used to grip a probe inserted. Optionally, the same probe inserter is used to remove probes. In one embodiment of the invention, insertion attachment 2144 acts as a lockable jaw (e.g., pliers-jaws) to engage a proximal side of the probe. In another embodiment, attachment 2144 includes an electro magnet which selectively engages the proximal side of the probe. Optionally, the proximal side of the probe (or connector 2143) is formed with a collar, other attachment design or a magnetic material, to support such selective locking. Optionally, attachment 2144 includes a vacuum connector to engage and/or positionally lock the probe by suction. In some embodiments of the invention, attachment 2144 includes an extension that fits in a groove formed in connector 2143 (or vice-versa). Optionally, attachment 2144 locks axially in place by rotation of connector 2143 relative to attachment 2144 (e.g., rotating one or both). Optionally, the groove is not axial (e.g., is circumferential or spiral). Alternatively or additionally, the grove includes an axial section for mounting connector 2143 onto the extension and includes a trans-axial groove section for preventing axial motion after the extension fits into the trans-axial groove section. The groove optionally has a profile which widens away form its surface, to prevent pulling out of the attachment form the groove, with the attachment including a matching widening tip.

In an exemplary use, the gripper is positioned around the probe and moved axially in the direction of the body until connector 2143 locks to or is engaged by attachment 2144. Then, actuator 2142 is reversed and the probed pulled out.

In an optional embodiment, gripper 2120 is moved in an X-Y plane, for example, using an X-Y translation mechanism and z-axis motion is provided by linear actuator 2142. One potential advantage of using a linear actuator, in x-y embodiments or even if gripper 2120 is on a robotic or otherwise movable arm, is that it may be easier and/or simpler to provide precise control of the insertion of the probe if its direction is held fixed by the gripper and motion in only one axis and degree of freedom of the placement system needs to be controlled.

Optionally, even if an inserter is used for inserting the probe, it may be removed by being grasped by gripper 2120 and pulled out. It is noted that, often, the positional accuracy control needed for pulling out a probe is less than that needed for pushing one in, as the tissue is somewhat flexible and the movement path is defined by the probe position in the tissue.

Optionally, gripper 2120 includes a position/mode where the jaws are close enough together to engage the probe and/or connector 2143. Optionally, the connector is sized to match the gripper. Other locking mechanism as described herein may be used as well. Optionally, element 2145 is designed to selectively narrow its lumen, for example, using a motorized iris mechanism, so as to selectively engage and release a probe. Optionally, the degree of movement is sufficient to selectively pass or engage connector 2143, if it is larger in diameter than the rest of the probe and optionally instead of releasing the jaws of gripper 2120. Optionally, element 2145 comprises a collet [SP] design with a ring that moves axially to radially compresses the collet lumen. In an alternative design, a shape-memory ring which can be heated to shrink the collet lumen, is provided.

In some embodiments, the probes are gripped and axially moved (towards and away form the body) by movement of the gripper. Locking mechanisms as described herein may be provided on the gripper.

In an exemplary embodiment of the invention, removed probes are reinserted. Alternatively or additionally, removed probes are placed in a quiver, optionally dropped into a quiver. Alternatively, the quiver may define fixed positions, optionally pre-determined, for example, defining a plastic matrix of positions or being formed of a relatively rigid sponge-like material. Before the procedure, some or all of the positions may be filled with probes, and the system programmed with their positions. When a probe is removed, it may be placed back in the quiver for later use.

In an exemplary embodiment of the invention, two arms are provided, one for probe insertion and one for probe removal. Additional insertion or removal arms ma be provided as well.

This ability has several important uses. System 2000 may be commanded to insert a plurality of cryoprobes according to a clinical treatment plan which includes a set of defined loci for insertion, cool selected probes to a selected extent, optionally heat selected probes (e.g. according to a cool/heat/cool clinical treatment protocol, or to facilitate disengagement of inserted cooled probes), and then return to re-grasp a selected inserted probe, and utilize inserter 2140 to reverse the probe insertion process and retract the inserted probe. Retraction may be complete, resulting in full retraction and removal of the inserted probe from patient 2001. System 2000, having inserted and cooled a plurality of probes, can optionally heat one or some or all of them and remove one or some or all of them.

Alternatively, retraction may be partial, according to a clinical protocol known in the art as a “pull-back” whereby an inserted probe is cooled, heated for disengagement, partially retracted, and then re-cooled at a selected partially-retracted position. Further alternatively, system 2000 can entirely retract one or more probes, and then re-insert them at additional defined loci for re-use there.

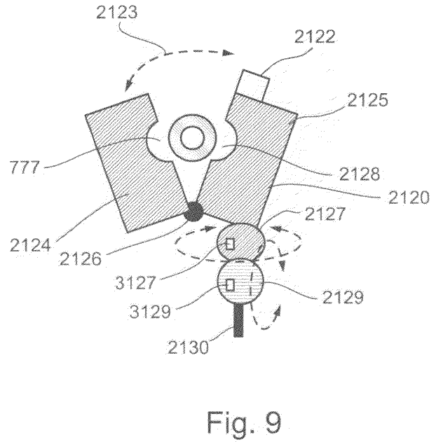

Attention is now drawn to FIG. 9, which is a simplified schematic of gripper 2120, according to an embodiment of the present invention.

Gripper 2120 comprises a stationary jaw 2125 attached to positioner 2130 and a moveable jaw 2124 movably connected to stationary jaw 2125, for example using a hinged connector 2126. When jaws 2125 and 2124 are close to each other, an insertion hole 2128 is formed between them. A cryoprobe 2010 or other probe may be inserted between jaws 2125 and 2124 and the jaws caused to approach each other, holding the inserted probe. Optionally, a probe bushing may be used to allow probes of different sizes to be used with gripper 2120.

In some embodiments, gripper 2120 is so designed that hole 2128 is configured as a probe guide sleeve 777 sized to enable passage of a probe therethrough, and deep enough to accurately direct movement of the probe in a desired direction toward a predetermined locus. In this embodiment, gripper 2120 serves as a probe guide similar to an individual aperture in Schatzberger's probe guide template, with the important difference that hole 2128 can be displaced freely and aimed at a selected angle, and there guide manual insertion of a template. Since gripper 2120 can be accurately aimed at a locus, with controller 2150 either providing movement commands to actuators of apparatus 2100 or else providing instructions and/or feedback to an operator according to knowledge of relative positions of gripper and locus in three-dimensional space, probe guide sleeve 777 can be aimed to approach a locus from a variety of angles, to avoid obstacles as necessary, and generally to facilitate manual probe insertion.

In a further alternative construction hole 2128 is sized to hold inserter 2130 at a desired position and direction, whence inserter 2130 can (manually or automatically) insert a probe.

To release an inserted probe, movable jaw 2124 is moved away from stationary jaw 2125 as depicted by dashed arrow 2123. Optionally, gripper 2120 may be embodied as a canister or equivalent mechanism for holding and releasing a plurality of probes.

Optionally, direction of probe insertion may be changed by one or two optional angular swivels 2127 and 2129 connected between gripper 2120 and positioner 2130.

Swivels 2127 and 2129 allow orthogonal rotations as depicted by dashed arrows in the Figure. In some embodiments swivels 2127 and/or 2129 are motorized and respond to movement commands communicated from controller 2150, enabling controlled automated angular approaches of cryoprobes inserted in gripper 2120 to patient 2001. In alternative embodiments, swivels 2127 and 2129 are not motorized but may be manually moved among plurality of preset positions. In further alternative embodiments swivels 2127 and 2129 are free to move, and comprise position sensors 3127 and 3129 operable to report angular orientations of swivels 2127 and 2129 to controller 2150.

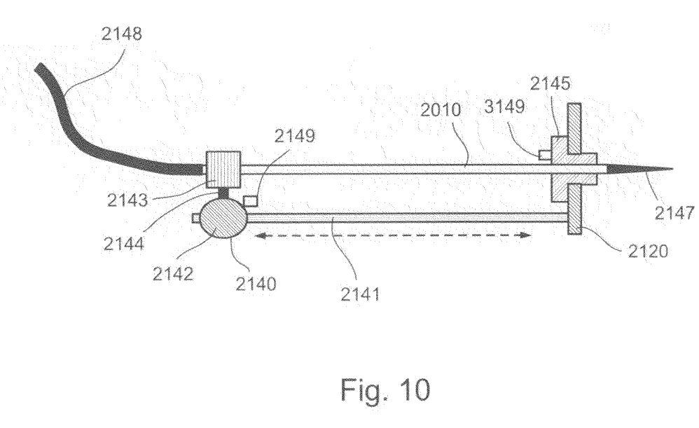

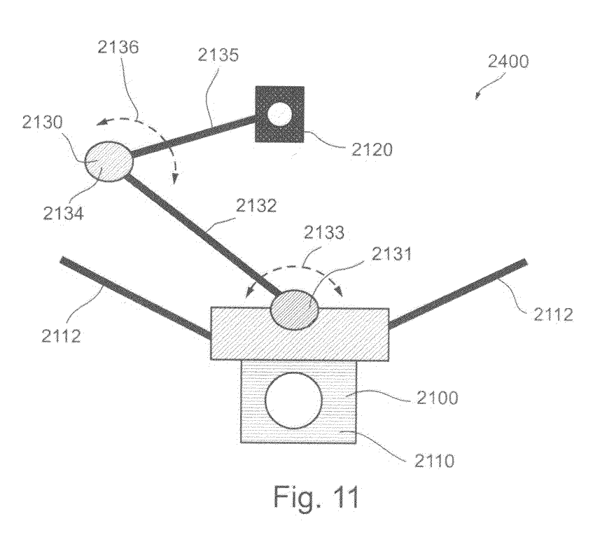

Attention is now drawn to FIG. 10, which is a simplified schematic of a probe inserter 2140, according to an embodiment of the present invention.

In an embodiment presented in FIG. 10 an insertion rail 2141 is connected to gripper 2120. Linear insertion actuator 1150 is movably connected to rail 1140 and is capable of motion 1151. A linear insertion actuator 2142 is attached to a hose-shaft connector 2143 by an insertion attachment 2144. A probe bushing 2145 is inserted into gripper 2120 and movably holds shaft 2146 of a therapeutic probe such as a cryoprobe 2010.

In operation, linear actuator 2142 moves toward gripper 2120, pushing sharpened probe tip 2147 towards and into body tissue, pulling flexible cryogen supply hose 2148 along with it.

Preferably linear insertion actuator 2142 is motorized and controlled by controller 2150. Alternatively, linear insertion actuator 2142 is not motorized and is manually operated. Whether or not actuator 2142 is motorized, actuator 2142 may comprise a positional sensor 2149 operable to report depth of insertion of probe 2010 into a body, which corresponds to the degree of advancement of probe 2010 through gripper 2120. If actuator 2142 is motorized, controller 2150 can cause probe 2010 to advance a desired distance. If actuator 2142 is not motorized, sensor 2149 can report depth of insertion to controller 2150, which can instruct a user to cease advancing probe 2010 when probe 2010 has reached a desired depth. Alternatively, depth of insertion may be monitored in real time by imaging modality 2218. Optionally, feedback based on algorithmic interpretation of resultant images can be used to control actuator 2142, or to inform a user how far to manually advance probe 2010.

As explained above, gripper 2120 and inserter 2140 can also be used to retract a gripped inserted probe from a body.

Optionally, inserter 2140 comprises a probe rotator 3149 which serves to rotate probe 2010 around its long axis during probe insertion, to facilitate penetration into tissues. Rotator may impart alternating short rotating motions, essentially twisting probe 2010 back and forth while inserting it, if probe 2010 has a connected cryogen input tube or other connection which would prevent free rotation of probe 2010.

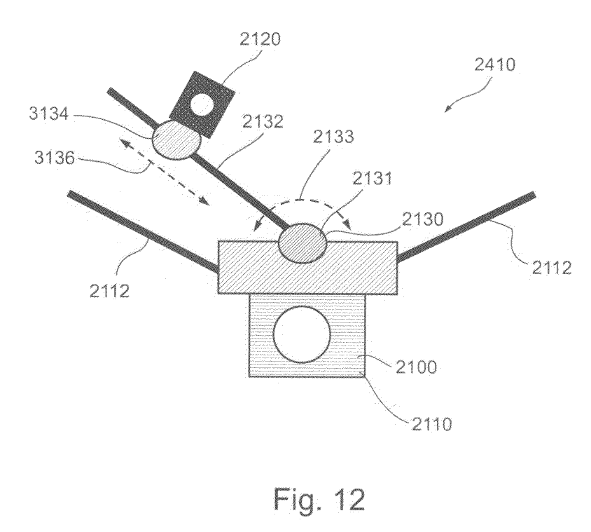

Attention is now drawn to FIG. 11, which is a simplified schematic of an optional configuration of positioner 2130, according to an embodiment of the present invention. FIG. 12 presents apparatus 211 embodied in a “dual angle” configuration here designated configuration 2400. Immobilizer 2110 is shown configured for attachment to a patient's bed (not seen) by means of bed connecting rods 2112. Between immobilizer 2110 and gripper 2120 positioner 2130 is shown in an exemplary embodiment which comprises a first angular actuator 2131 actuating first rigid member 2132 through angular motion arc 2133, and a second angular actuator 2134 connected to first rigid member 2132 and actuating a second rigid member 2135 through angular motion arc 2137. Actuators 2134 and 2131 may comprise motors whose motion is commanded by controller 2150 and is operable to position gripper 2120 as desired. Alternatively, actuators 2134 and 2131 may comprise sensors 2138 and 2139 operable to report positions of actuators 2134 and 2131.

In operation, if actuators 2134 and 2131 are motorized, controller 2150 may issue commands to move gripper 2120 to a desired position. If actuators 2134 and 2131 are not motorized, arms 2132 and 2135 may be moved by a user, with controller 2150 receiving information enabling it to calculate the position of gripper 2120 and to issue instructions to a user for moving gripper 2120 into a desired position.

Attention is now drawn to FIG. 12, which is a simplified schematic of a “polar angle” configuration of positioner 2130, here designated configuration 2410, according to an embodiment of the present invention.

Configuration 2410 is similar to configuration 2400, differing therefrom in that a second angular actuator 2134 and rigid member 2135 are here replaced by a linear actuator 3134, enabling motion of actuator 3134 as indicated by arrow 3136. Features and functionality of apparatus 2100 in configuration 2410 are otherwise similar to those presented with respect to configuration 2400.

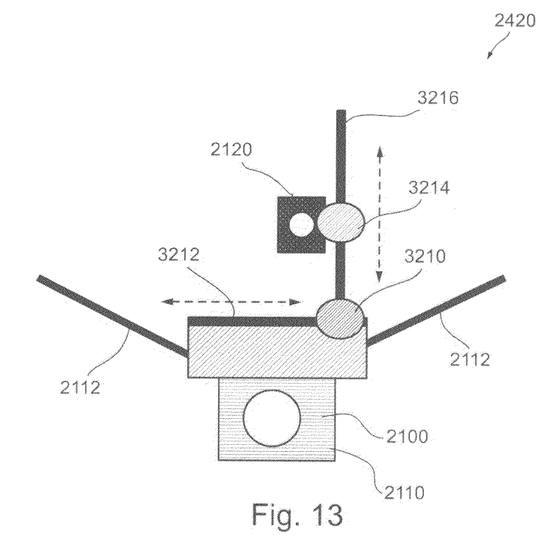

Attention is now drawn to FIG. 13, which is a simplified schematic of a “Cartesian” configuration of positioner 2130, here designated configuration 2420, according to an embodiment of the present invention.

Configuration 2420 is similar to configuration 2400, differing therefrom in that a first and second angular actuators 2131 and 2134 are here replaced by linear actuator 3210 operating along rigid member 3212 and by linear actuator 3214 operating along rigid member 3216. Features and functionality of apparatus 2100 in configuration 2420 are otherwise similar to those presented with respect to configuration 2400.

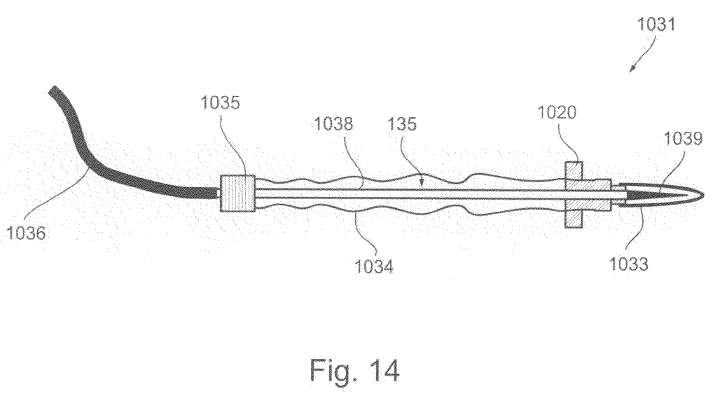

Attention is now drawn to FIG. 14, which is a simplified schematic of a sterilization cover for a probe, according to an embodiment of the present invention.

FIG. 14 presents a sterilization-maintaining probe 1031 for use with system 2000. Probes inserted in the body must be sterile. A removable cover 1033 is connected to bushing 1020 to maintain sterility of tip 1039 during handling of probe 1031, installation of probe 1031, etc. Similarly, tubular flexible cover 1034 connected between bushing 1020 and hose-shaft connector 1035 keeps shaft 1038 sterile while sterilization maintaining probe 1031 is manipulated. Optionally, gripper 2120 can be sterilized, therefore once sterilization maintaining probe 1031 is inserted into a sterilized gripper 2120, cover 1033 may be is removed to expose sharpened tip 1039, and shaft cover 1034 can be moved or removed if needed.

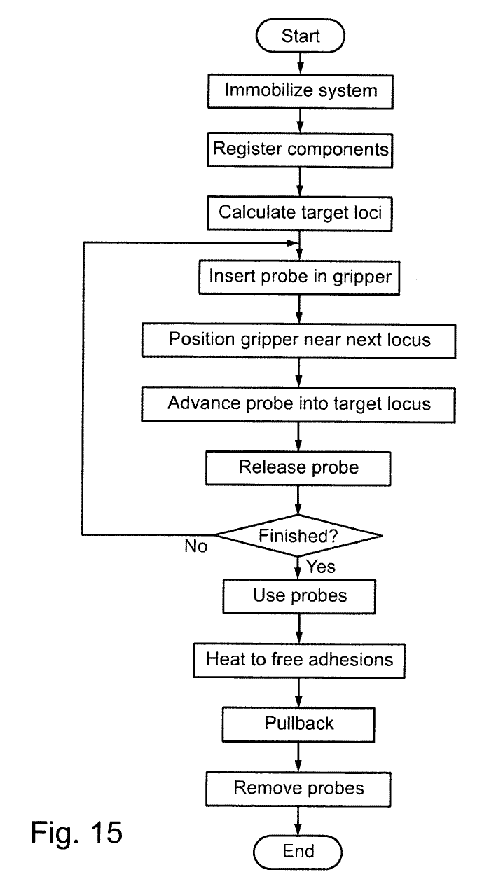

Attention is now drawn to FIG. 15, which presents a simplified flowchart of a cryosurgery method, according to an embodiment of the present invention.

Apparatus 2100 is immobilized with respect to a patient and preferably also an imaging modality, enabling to register patient, image source, images, and apparatus 2100 in a common coordinate system. A user can input loci for probe insertion, or system 2000 or another system can calculate them based on information contained in registered images or input by a user.

Once a set of insertion target loci are known, system 2000 undertakes an iterative process comprising inserting a probe in a gripper, positioning the gripper near a selected insertion target locus, advancing the probe into the locus, releasing the probe, and repeating that iterative process for all the probes to be inserted.

Once probes are inserted they are used. Cryoprobes, for example, are typically cooled to ablate tissue. Optionally, probes are heated to free adhesions, and apparatus 2100 is optionally used to perform probe pullback as described above, and to remove probes at end of treatment.

It is to be noted that positioning of gripper 2120 near a locus and angled to point towards that locus may be done automatically by motorized actuators controlled by controller 2150, or may be done manually with position sensors or other sensors reporting to controller 2150, which can issue instructions to a user based on received sensor information.

Advancing a probe for insertion towards a locus, and retraction of a probe from a locus, may also be done automatically or manually.

As described hereinabove, target loci can be input by users or calculated by elements of system 2000 or associated systems. That process produces a set of locations in three-dimensional space where probes are to be inserted.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A cryosurgery system comprising at least one cryoprobe and a positionable element adapted to at least guide insertion of said probe into a body of a patient.

2. The system of claim 1 further comprising a probe insertion apparatus operable to sequentially insert a plurality of cryoprobe operating tips into a patient at predefined loci within the body of said patient.

3. A probe insertion guide comprising
(a) a moveable probe guide sleeve;
(b) a positioner for positioning said probe guide sleeve; and
(c) a controller operable to receive a definition of a locus within a body of a patient and to direct positioning and orienting of said probe guide sleeve so that a probe advanced through said sleeve will advance in direction of said locus.

4. The probe insertion guide of claim 3, wherein controller directs said positioning and orienting by sending movement commands to motorized actuator components of said positioner.

5. The probe insertion guide of claim 3, wherein controller directs said positioning and orienting by communicating guidance information to a user.

6. A probe insertion system comprising:
(a) a plurality of therapeutic probes; and
(b) a probe insertion apparatus for sequentially inserting said plurality of cryoprobes into a patient, said apparatus comprises
(i) an immobilizer for maintaining said apparatus in a fixed spatial relationship to a patient;
(ii) a probe gripper;
(iii) a positioner for positioning said grasper with respect to said patient; and
(iv) an inserter operable to linearly advance a probe grasped by said grasper.

7. The system of claim 6, wherein a majority of said plurality of therapeutic probes are cryoprobes.

8. The system of claim 6, wherein said plurality of therapeutic probes comprises at least four cryoprobes.

9. The system of claim 6, wherein said immobilizer further comprises a rectal ultrasound attachment for attaching said apparatus to a rectal ultrasound probe.

10. The system of claim 6, further comprising a controller which comprises a command module operable to calculate and communicate to said apparatus a command sequence commanding said apparatus to insert a distal end of a therapeutic probe grasped by said grasper into a locus within a body of said patient.

11. The system of claim 10, further comprising a memory for receiving definitions of a plurality of loci defined within said body of said patient, said command module being operable to calculate and communicate to said apparatus a plurality of command sequences each commanding said apparatus to insert a distal end of a therapeutic grasped by said grasper into one of said plurality of loci.

12. The apparatus of claim 10, wherein each of said command sequences comprises
(a) a set of positioner commands commanding movement of said grasper towards one of said loci; and
(b) a set of inserter commands commanding said inserter to advance said grasped cryoprobe towards said one of said loci.

13. The system of claim 10, further comprising a locus-defining module operable to define said plurality of loci based on at least one image received from an imaging modality.

14. The system of claim 13, wherein said locus-defining module comprises a user interface for receiving user input identifying a treatment target with respect to said received image.

15. The system of claim 13, wherein said locus-defining module comprises a processor programmed to identify a treatment target based on information in said received image.

16. The system of claim 10, wherein said apparatus comprises a plurality of position sensors operable to report positions of apparatus components to said controller.

17. The system of claim 10, wherein said apparatus comprises at least one motorized actuator operable to respond to a command received from said controller.

18. The system of claim 6, wherein said inserter comprises a rotation module operable to rotate a grasped cryoprobe around its long axis while advancing said grasped cryoprobe.

19. The system of claim 18, wherein said rotation module is operable to periodically alternate direction of rotation of said grasped cryoprobe during advancement of said grasped cryoprobe.

20. The system of claim 6, wherein said grasper comprises a probe-type sensor operable to report a type of a probe.

21. The system of claim 10, further comprising a therapeutic probe without cooling capacity.

22. The system of claim 21, comprising a probe selected from a group consisting of a heating probe and a thermal sensor probe.

23. The system of claim 21, wherein at least one of said plurality probes comprises a marking identifying probe-type of said at least one probe, and said gripper comprises a probe-type detector for detecting type of a probe grasped by said gripper.

24. The system of claim 21, wherein said calculation of command sequences varies as a function of detected probe-type.

25. The system of claim 6 wherein said positioner has a configuration selected from a group consisting of a dual angle configuration, a polar angle configuration, and a Cartesian configuration.

26. The system of claim 12, wherein said controller is programmed to receive a locus definition from a user.

27. The system of claim 26, wherein said controller is programmed to calculate a locus definition based on information received from a user.

28. The system of claim 12, wherein said controller is programmed to calculate a locus definition based on an image received from an imaging modality.

29. The system of claim 10, wherein said controller is further programmed to control heating and cooling of said plurality of cryoprobes.

30. The system of claim 10, wherein said controller comprises programming for calculating and communicating a command sequence directing said positioner to position said gripper at a cryoprobe previously inserted by said apparatus.

31. The system of claim 30, wherein said gripper is operable to grip an inserted cryoprobe when so commanded by said controller, and said controller comprises programming for commanding said gripper to grip an inserted cryoprobe when said gripper is positioned at a previously inserted cryoprobe.

32. The system of claim 31, wherein said controller is further programmed to individually control heating and cooling of said plurality of cryoprobes.

33. The system of claim 32, wherein said controller is operable to command insertion of a first cryoprobe followed by first cooling of said first cryoprobe followed by insertion of a second cryoprobe followed by repositioning of said positioner at said inserted first cryoprobe followed by re-grasping of said first cryoprobe.

34. The system of claim 33, wherein said controller is further operable to command removal of said first cryoprobe from said patient.

35. The system of claim 34, wherein said controller is further operable to command insertion, use, and removal of said first cryoprobe, followed by reinsertion of said first cryoprobe at an additional locus.

36. The system of claim 34, wherein said controller is further operable to command removal of all inserted cryoprobes from a patient.

37. The system of claim 33, wherein said controller is further operable to insert a cryoprobe to a first position, cool said probe, warm said probe, partially retract said probe to a second position, and cool said probe at said second position.

38. The system of claim 12, wherein said positioner comprises a swivel module operable to aim said gripper at a variable angle with respect to said patient.

39. The system of claim 38, wherein said controller is operable to command insertion of a cryoprobe into said patient at a selected angle.

40. The system of claim 39, wherein said controller comprise an obstacle-avoidance module operable to command insertion of a cryoprobe at an angle calculated by said module to avoid approaching an obstacle at a defined position within said patient.

41. The system of claim 6, further comprising a cryoprobe introducer usable to insert at least a subset of said plurality of cryoprobes into said patient in a common insertion.
